(19) United States
(12) Patent Application Publication
(10) Pub. No.: US 2026/0273064 A1
(43) Pub. Date: Sep. 17, 2026

(54) Fermenting pharmaceuticals to reduce adduct risks coupled with functional enzyme metrics

(71) Applicant: Kenneth Raymond Quan, Poway, CA (US)

(72) Inventor: Kenneth Raymond Quan, Poway, CA (US)

(21) Appl. No.: 19/567,276

(22) Filed: Mar. 16, 2026

Related U.S. Application Data
(60) Provisional application No. 63/772,577, filed on Mar. 16, 2025.

(51) Int. Cl. A61K 47/46; A61K 45/06; A61K 47/18; A61K 47/22; A61K 47/26; A61K 47/42; C12M 1/00; C12M 1/34; C12M 3/00

(52) U.S. Cl. CPC A61K 47/46; A61K 45/06; A61K 47/183; A61K 47/22; A61K 47/26; A61K 47/42; C12M 23/44; C12M 41/06; C12M 41/12; C12M 41/26; C12M 41/30; C12M 41/34

(57) ABSTRACT

Disclosed are kits, methods, systems, and devices for reducing mutagen-related biomolecular adduct risk during pharmacotherapy. A therapeutic agent comprising an active pharmaceutical ingredient or a therapeutic supplement is combined with one or more adjunct components selected from probiotics, antioxidants, buffering agents, chelators, plant-derived materials, algae-derived materials, enzymes, vitamins, and combinations thereof to form a mixture that is fermented prior to oral or topical administration. Biomarker data comprising one or more adductomic endpoints is obtained from a subject and compared with functional protein metrics and comparator data, thus subsequent preparation cycles are adjusted by modifying one or more fermentation parameters including duration, temperature, pH, light exposure, concentration, and/or order of adjunct addition. Clinical, pharmacokinetic, pharmacodynamic, and/or functional protein metrics are used to guide adjustment. System embodiments include one or more kits, compositions, vessels, and instructions configured to output updated preparation instructions while maintaining intended therapeutic efficacy.

### CROSS-REFERENCE TO RELATED APPLICATIONS

This submittal claims priority to U.S. Provisional Patent Application No. 63/772,577 filed on Mar. 16, 2025, the contents of which are incorporated herein by reference in their entirety.

### STATEMENT REGARDING FEDERALLY SPONSORED RESEARCH OR DEVELOPMENT

No federally sponsored research or development was used in the conception or reduction to practice of the invention disclosed in this application.

### FIELD OF THE INVENTION

The present invention relates generally to pharmaceutical compositions, fermentation of combinations, and therapeutic adduct monitoring approaches for attenuating the absorption of mutagenic compounds in human and veterinary subjects. The invention further relates to subject- or cohort-specific drug delivery configurations that reduce the formation of electrophilic metabolites and intermediates, and ultimately, the associated biomolecular adducts during pharmacotherapy and dietary administration.

The invention relates specifically to orally and topically administered formulations, combinations, and associated methods that reduce, neutralize, or otherwise mitigate the formation, bioavailability, and/or biological activity of mutagenic substances arising from pharmaceutical and/or food storage, shipment, handling, patient administration, digestion, and/or metabolism.

### BACKGROUND OF THE INVENTION

### Mutagenic Risk in Pharmaceutical Therapy

A mutagen is a substance that causes or leads to a mutation or an adduct (covalent modifications) in the DNA of a cell. DNA changes (also detected via adductomics) may harm cells and lead to diseases such as cancer as described by the National Cancer Institute (NCI) at cancer.gov. For the purpose of this disclosure, a mutagen is also defined as a substance that causes or leads to protein adducts. Covalent adducts on proteins and DNA can damage multiple classes of biomolecules and tissues in a variety of ways.

For one mutagen, nitrosamines, metabolism is considered part of the Earth's nitrogen cycle and can cause these DNA changes and adducts. Specifically for nitrosamines, DNA changes represented as covalent adducts arise from the precursor's amine functional group activation. Nitrosamines are metabolized into the electrophilic diazonium cation, and then an adduct may form in vivo. Such adduct formation has been implicated in carcinogenic risk and regulatory safety concerns. This covalent DNA adduct generation is considered the cause of tumorigenesis and is now evaluated as a central dogma of chemical carcinogenesis, as described by Stiborová, M. in “Formation of Covalent DNA Adducts . . . ”, J. Vis. Exp. (133), e57177 (2018). DOI: 10.3791/57177.

### Limitations of Related Art

Numerous pharmacological agents undergo metabolic activation, resulting in the formation of reactive electrophiles capable of forming covalent adducts with proteins, nucleic acids, and other biomolecules. This activation or nitrosation can also occur under conditions related to the manufacturing process for the drug product. An example of this is the reaction with residual nitrites in the drug product, as described by the FDA's Control of Nitrosamine Impurities in Human Drugs: Recommended Acceptable Intake Limits for Nitrosamine Drug Substance-Related Impurities (NDSRIs). Specifically, NDSRIs can form by reacting with APIs and API fragments that contain secondary, tertiary, or quaternary amines (these amines are common functional groups in many pharmaceuticals) and nitrites. Nitrites are produced by oral and intestinal bacteria and ingested through certain foods, making risk mitigation difficult. The FDA has published recommended acceptable intake limits for nitrosamine impurities and a table of hypothetically at-risk NDSRIs (Table 1, periodically updated). With over 280 NDSRIs, the FDA makes guidance on quality control limits, but this is a discrete mitigation in an environment with ubiquitous nitrites and nitrosation. These resolutions have trade-offs and are not patient-specific, resulting in a persistent risk of mutagen exposure for many populations.

Thus, many pharmaceuticals have mutagens that reside or are reasonably expected to reside in the final drug substance as determined by the FDA pharmaceutical guidance ICH M7(R2). Since nitrosamines are part of the continuous nitrogen cycle, these impurities may increase during chemical synthesis, formulation, drug shipment, storage, co-administration with foods, and/or drug metabolism within the body. These strategic limits and controls are protective, but they do not totally eliminate exposure.

Various tactics have been proposed and utilized to mitigate nitrosamine formation in pharmaceutical products, including formulation and process alterations that rely on antioxidants and pH modification. For example, FDA-published guidance on mitigation strategies describes literature-based approaches in which commonly used antioxidants (e.g., ascorbic acid (vitamin C) and α-tocopherol (vitamin E)) and/or pH adjustments may inhibit nitrosamine formation during manufacture, storage, and under conditions relevant to the gastrointestinal environment. A limitation of these approaches is that inclusion of antioxidants may be formulation-dependent and may not be practical or cost-effective for all drug products and storage requirements, nor tailored to patient-specific needs. In addition, antioxidants may be clinically unsuitable in certain contexts due to potential drug-excipient interactions (e.g., interactions with vitamin C and medicines such as propranolol or flecainide). Thus, these strategies are not integrated into most drug products nor into patient-specific formulations.

This present invention provides drug-delivery systems and formulation methods designed to reduce mutagenic risk while managing clinical compatibility issues and interaction considerations. This invention is efficient because it can be applied to numerous medications yet is readily customizable to fit any patient-specific condition, potentially resulting in decreased adducts, reduced cancer risk, and clinically insignificant side effects regardless of any pre-existing patient-specific factors.

In conclusion, current strategies to control mutagenic risk primarily focus on upstream manufacturing controls, including selection of low-impurity raw materials, process optimization to minimize formation of reactive species, and analytical testing and batch rejection. While effective to a degree, these approaches do not totally eliminate mutagens, nor do they address in vivo formation of mutagenic compounds occurring during digestion, metabolism, or interaction with gut microbiota. Furthermore, existing pharmaceutical products are rarely designed to actively neutralize or mitigate mutagenic species after administration in vivo.

### Unmet Need in Healthcare

Standing alone, adductomic measurements may be difficult to standardize and interpret in a clinically meaningful manner. Such difficulty may arise from limited harmonization of analytical methods and reference frameworks, difficulty attributing measured adducts to particular sources or mechanisms, and uncertainty regarding the clinical significance of lowering a measured adduct burden without a corroborating functional readout or demonstrable benefit. Accordingly, a need remains for a framework that improves the interpretability, comparability, and clinical utility of adductomic data. In certain embodiments, one or more functional protein metrics, as defined herein, are evaluated together with one or more adductomic endpoints to provide a corroborative functional context for measured changes in adduct burden and to assist determination of whether such changes are associated with a clinically meaningful biological response. Ultimately, this may increase the practical and clinical value of adductomic monitoring through the iterative PMM/PMD optimization described herein.

The present disclosure addresses this need by evaluating adductomic endpoints together with one or more functional protein metrics. In certain embodiments, longitudinal changes in adduct burden are assessed in relation to corresponding changes in one or more functional protein metrics and, where appropriate, matched-cohort comparator data, subject-specific baseline data, and/or prior response patterns. This correlation may provide a more standardized and clinically meaningful basis for determining whether a decrease in adduct burden is associated with improved biologic function. By way of non-limiting example, a trend toward a more typical hemoglobin P50 value occurring together with a reduction in hemoglobin adduct burden may provide a quantitative correlation between improved hemoglobin performance and decreased adduction. Similar concordance-based assessment may be applied to HbA1c discordance, INR, whole-blood clotting time, ferritin/hemoglobin relationships, and other functional protein metrics, thereby increasing the practical value of adductomic monitoring in iterative PMM/PMD optimization.

Prior mitigation techniques generally fail to account for the temporal dynamics of reactive metabolite formation, inter-individual metabolic variability, or the mechanistic coupling between primary therapeutic agents and adduct-suppressing co-therapies. As a result, existing solutions have not achieved reliable, scalable, or clinically practical and efficient reductions in electrophilic mutagen exposure. Accordingly, there is a need for pharmaceutical formulations and therapeutic protocols that proactively mitigate these mutagen risks after medicines are furnished and while the medicine is being metabolized in vivo. The invention described herein addresses this gap by coordinating clinical delivery of therapeutic agents with adduct-mitigating compounds. This invention utilizes agents that scavenge electrophilic intermediates (mutagens), inhibit nitrosation, and/or reduce pro-mutagenic conditions. The proposed invention is a system and protocol that seeks to reduce formation of both DNA and protein adducts and functions independently of upstream manufacturing controls, is compatible with numerous drug products, and can be individualized based on patient-specific factors. Thus, the design is adaptable: components can be tailored to different drug classes, dietary patterns, animal species, humans, medical histories, and drug strengths. This invention also allows for synchronized dosing and customization to counteract patient- or cohort-specific metabolic pharmacodynamic (PD) and pharmacokinetic (PK) issues and side effects. Such patient-specific, mechanistically informed approaches could significantly lower mutagen exposure, adduct formation, and disease risks while preserving therapeutic efficacy, fulfilling the unmet need. Adductomic endpoints and functional protein metrics, as defined in Section 8.1, provide a biomarker framework for monitoring subject response and guiding iterative adjustment of the PMM and/or PMD. The present disclosure differs from existing approaches in that mutagen mitigation is performed ex vivo, at or near the point of administration, through fermentation carried out prior to administration of the therapeutic agent.

### SUMMARY OF THE INVENTION

The disclosed embodiments and summaries are provided for purposes of illustration and are not intended to limit the scope of the present disclosure. Variations, modifications, substitutions, and combinations of the described embodiments will be apparent to those of ordinary skill in the art and are intended to be included within the scope of the present disclosure and the appended claims.

The present disclosure differs from existing approaches in that mutagen mitigation is performed ex vivo, at or near the point of administration, by fermenting a therapeutic agent prior to administration and then iteratively adjusting composition, fermentation parameters, and/or regimen variables based on adductomic endpoints and, in some embodiments, one or more functional protein metrics, as defined herein.

### 6.1 Field and Problem

This invention addresses the growing concern about mutagenic impurities in pharmaceuticals, diet, and the exposome, particularly nitrosamine drug substance-related impurities (NDSRIs) and other mutagens that form during food and drug manufacture, storage, shipment, or in the gastrointestinal tract. The exposome is the sum of all exogenous and endogenous exposures experienced by an individual over a lifetime. For medicines, nitrosamines may arise from reactions between residual nitrites in excipients and secondary or tertiary amine groups present in drugs and foods. Per FDA guidance, there are numerous hypothetical NDSRIs, yet current mitigation efforts are primarily upstream and do not neutralize mutagens once a medicine is ingested. A need remains for a proactive system, product, and protocol to reduce mutagen exposure.

### 6.2 Overview of the Invention

Described herein are systems, protocols, compositions, and methods for reducing exposure to nitrosamines and other mutagens, and for mitigating associated risk, arising from a subject's exposome, pharmacotherapy, and diet.

For the purpose of the disclosure, the terms “subject”, “user”, and the “patient” are used interchangeably and refer to the same consumer, customer, buyer, or end user.

The invention provides a system and protocol comprising the integrated Probiotic Medicine Mixture (PMM) and an optional Probiotic Medicine Dosing System (PMD), all designed to neutralize or attenuate mutagen exposure in vivo while preserving therapeutic efficacy in pharmacotherapy. Described herein are compositions and methods for reducing nitrosamine impurities and mutagen exposure risk. The invention provides a system and protocol for fermenting marketed medications with selected adjunct components to reduce mutagenic risk and improve clinical outcomes. The PMM is formulated on a patient- or cohort-specific basis and may include detailed instructions for the patient or caregiver on how to combine and use the components. The mixture is fermented with the patient's existing medicines for a patient- or cohort-specific period, thereby allowing for a minimization of the systemic absorption of mutagens. The system and design of the invention (the PMM, PMD) may utilize PK and PD parameters, fermentation timing, users' diet, and PMM customizations. The invention may also incorporate a separate but also tandem execution of comprehensive medication management (CMM) or medication therapy management (MTM) to create a cost-effective and efficient personalized and exhaustive risk-mitigation strategy either at the patient- or cohort-specific level. Lastly, the design of the invention allows for continuous periodic monitoring and adductomics on the collected users' and patients' bio specimens (for example, blood samples).

### 6.3 Key Components

### Probiotic Medicine Mixture (PMM)

In some embodiments of the PMM, comprises two or more of the following main components:
1. Vitamins and/or supplements (Component 1)—Potential examples include vitamin C and vitamin E. These antioxidants can inhibit nitrosation and scavenge electrophilic intermediates and nitrites.
2. GRAS Plant or Algae Substances (Component 2)—Food-grade plant materials and derivatives thereof, including plants from the orders Ericales, Rosales, Brassicales, Poales, Asparagales, Cucurbitales, Caryophyllales, Asterales, Lamiales, Malpighiales, Fagales, Fabales, Laurales, and/or Pinales may be used. In some embodiments, such plant-derived materials provide nucleophilic and/or antioxidant constituents that can bind, neutralize, or otherwise attenuate mutagens and/or reactive intermediates. Lastly, in certain embodiments, the Brown algae (e.g., class Phaeophyceae, phylum Ochrophyta), red algae (phylum Rhodophyta), and green algae (e.g., phyla Chlorophyta and/or Charophyta) may also be utilized as a component 2.
3. Probiotics (Component 3)—Yeasts of the order Saccharomycesles and/or bacteria from the class Actinobacteria, Bacilli, and/or phylum Cyanobacteria. Probiotics maintain healthy gut flora and may metabolize or sequester mutagens.
4. Probiotic Nutrients, Prebiotics, and/or Excipients (Component 4)—Sugars such as dextrose, glucose, sucrose, and maltose. These support probiotic viability and accelerate the fermentation process.
5. The Active Pharmaceutical Ingredient (API) or patient's standard medication (Component 5)—The patient's prescribed or over-the-counter drug(s). In certain aspects of the invention, the practitioner or provider may select and adjust the relative proportions and combinations of the recited components to accommodate patient-specific conditions, adherence patterns, and potential interactions. Any subset of Components 1 through 4 may be combined with Component 5 (the API) in the PMD in accordance with clinical monitoring, patient allergies, or practitioner/patient preferences. In some embodiments, any combination of the five components is provided in a single dosage form (for example, a capsule or tablet), and fermented in the PMD with water.

### 6.4 Probiotic Medicine Dosing System (PMD)

The PMD is an organizer and fermenter that facilitates patient adherence and ensures appropriate fermentation. It includes a temperature monitor and a light source (customized to any specific wavelength) that can be customized to the patient's adrenergic feedback. The PMD holds the PMM components and provides controlled fermentation conditions. In one embodiment, the key feature of the PMD is that its design of component 1-4 is specific to a specific patient and certain drug regimen. Of note, the invention can also comprise an alternative embodiment, where a single PMM capsule may be utilized with the PMD or similar device and fermented, thus allowing for a convenient single-action combination.

### 6.5 General Method of Operation

As deemed appropriate by the provider, the methods and systems described herein may be tailored or altered at a patient-specific and/or cohort-specific level. In certain embodiments, a pharmacist or provider may independently configure the PMM compositions and associated PMD operating parameters, provided that at least one supplement plant, probiotic, or prebiotic element is included.

Broadly, in one embodiment, the method may include selecting and configuring any combination of Components 1-5, including one or more fermentation parameters such as fermentation time, illumination wavelength, and fermentation temperature, as dictated by clinical assessment. In some embodiments, an iterative or continuous-improvement framework is implemented in which adjunct selection, composition, and/or dosing parameters are adjusted based on analysis of current and historical adductomic monitoring data and clinical response. In certain embodiments, the method further includes providing patient- or caregiver-facing instructions, including a defined order of operations, to support correct preparation and consumption.

For illustrative purposes, one embodiment and exemplary method comprises administering Losartan Potassium 25 Mg Tablet (Component 5) in combination with Saccharomyces boulardii (Component 3) according to a provider and patient-centered care plan. The plan was developed using clinical assessments and biomarker data. Furthermore, in this exemplary method, the plan developed a fermentation duration of approximately 1 minute at a temperature range of about 28-30° C.

More generally, the disclosure provides flexibility for a provider to adjust composition and/or operating parameters of the PMM and PMD, including component identity, component ratios, fermentation time, temperature, and/or light exposure, without departing from the underlying concept.

In one embodiment, the PMD can be prepared using EPA- or FDA-approved drinking water (optionally alkaline). The user, provider, or pharmacist may prepare one or more plant-derived components (e.g., by comminution or blending) and combine the plant component(s) with water. A fermenter may be filled with said water mixture, which may be maintained at about 28-33° C. or otherwise controlled at a temperature selected by the provider. Lastly, one or more PMM components (Components 1-5) are introduced into the fermenter. In this exemplary method, the fermentation is conducted for approximately 1 to 3 minutes according to a patient-specific or cohort-specific PMD plan. The patient then consumes the resulting fermented mixture and/or a prebiotic mixture. In certain embodiments, controlled fermentation, probiotics, and plant constituents provide bio-nucleophiles and antioxidants that may react with electrophilic mutagens and/or mutagenic reactive intermediates, thereby reducing mutagenic exposure risk while maintaining adequate bioavailability of the medicine for pharmacotherapeutic benefit. As noted, the disclosure provides flexibility for a provider or pharmacist to alter operating parameters of the PMM and PMD, thus fermentation times may vary by patient, regimen, and therapeutic agent; alternative embodiments include fermentation times from approximately 1 minute to about 24 hours and other ranges, shorter or longer as discussed herein.

In all embodiments described herein, the therapeutic agent (Component 5) is fermented ex vivo, outside the body, and prior to administration to the subject. Embodiments in which the therapeutic agent is not fermented prior to administration are excluded from the claimed invention.

For this disclosure, herein it is established that after administration of the fermented therapeutic agent, biomarker data comprising one or more adductomic endpoints and, in some embodiments, one or more functional protein metrics is obtained longitudinally and/or according to a monitoring cadence within a close loop. This continuously iterative adjustment loop is then executed to modify one or more PMM composition variables, PMD operating parameters, fermentation conditions, and/or regimen variables in order to drive the biomarker data toward one or more predefined target values. In non-limiting embodiments, such target values include an HSA adduct burden of about 0.1 μmol adduct per mg HSA or less, a hemoglobin P50 of about 25 mm Hg to about 27 mm Hg, and/or a discordance between HbA1c-derived average glucose and directly measured blood-glucose averages of not more than about 7%.

### 6.6 Advantages and Novelty of the Invention

The PMM can be designed to mitigate mutagen exposures. In this embodiment, the PMM ultimately combines and ferments strategic bio-nucleophiles (from plants and probiotics) and antioxidants (from plants, vitamins, and supplements) into the same mixture as component 5. The PMM mixture aims to convert or sequester electrophilic mutagens while inhibiting nitrosation and other deleterious conversions, thereby reducing mutagen exposure in vivo.

The PMM can be customized according to the patient's medical and pharmaceutical history, diet, and clinical needs. The optional PMD may assist adherence and allow real-time fermentation control, while an alternative one-capsule or kit option simplifies fermentation.

In many embodiments, the therapeutic agent is fermented ex vivo together with one or more selected adjunct components. In other embodiments, one or more adjunct components are first fermented ex vivo, and the therapeutic agent is subsequently fermented ex vivo with the adjunct-containing composition, or the therapeutic agent is first fermented ex vivo and then combined with one or more separately fermented adjunct components, provided that the therapeutic agent itself is fermented ex vivo prior to administration.

In certain embodiments, the PMM framework described herein enables flexible selection and adjustment of components, concentrations, and (where applicable) fermentation or processing parameters. The embodiments presented herein are illustrative and represent only a subset of a broader set of permissible permutations supported by this disclosure. In some implementations, clinicians and pharmacists may tailor the identity and dosing of mixture components without departing from the underlying concept of reducing mutagenic risk. This flexibility may allow the PMM approach to be applied across diverse drug classes and patient populations, including adjustment based on a patient's or cohort's adductomic profile and/or continuous monitoring data and history, thereby accommodating a range of clinical scenarios. In contrast to approaches that require drug-by-drug modifications to manufacturing controls, certain embodiments described herein are configured for use at or near the point of care or point of administration. This may be achieved by combining marketed therapeutics with selected adjuncts (Components 1 to 4), thus providing a practical, patient-centered strategy to manage mutagenic impurities and related reactive intermediates at the site and time of administration.

Following administration of the fermented PMM prepared using the PMD, biomarker-guided iterative adjustment is performed using adductomics and, in some embodiments, one or more functional protein metrics. This integrated ex vivo fermentation and post-administration biomarker-feedback architecture provides a subject-specific and/or cohort-specific method for reducing mutagen-related adduct burden, improving corroborative functional protein metrics, and optimizing PMM composition, PMD parameters, and regimen execution in view of the subject's exposome, therapeutic regimen, and clinical response.

The present invention offers an innovative approach to mitigating mutagenic exposure during pharmaceutical and dietary therapy while measuring adductomics. By incorporating or fermenting components that neutralize, sequester, or inhibit the formation of mutagenic species, the invention provides a scalable solution applicable across a wide range of therapeutic areas. The compositions and methods are designed for flexibility, allowing for patient-specific or cohort customization and compatibility with existing drug products. This technology has the potential to enhance long-term patient safety, reduce health risks, and address a critical mutagen gap in human and veterinary healthcare.

### BRIEF DESCRIPTION OF THE DRAWINGS

FIG. 1 illustrates an exemplary embodiment of a probiotic medicine dosing system (PMD) including an organizer (System 100) and a fermenter vessel configured to ferment a composition comprising a therapeutic agent and one or more adjunct components, wherein the fermenter includes a temperature monitoring component and a light source configurable to emit one or more wavelengths.

FIG. 2 depicts an exemplary decision workflow and feedback loop (System 888) for configuring a probiotic medicine mixture (PMM) composition and continuously adjusting one or more components, and PMD preparation parameters based on adductomics, functional protein metrics, and/or nitrite testing, in coordination with MTM/CMM clinical assessment and follow-up measurement intervals.

### DETAILED DESCRIPTION OF THE DISCLOSURE

### 8.1 Definitions

For purposes of this disclosure, the following terms have the meanings set forth below. Unless the context indicates otherwise, the terms “subject,” “patient,” and “user” may be used interchangeably.

“Adduct” refers to a covalent modification of a biomolecule, including without limitation a DNA adduct and/or a protein adduct.

“Therapeutic agent” refers to an agent administered to a subject for a therapeutic purpose and includes, without limitation, (i) an active pharmaceutical ingredient (API); (ii) a marketed or final drug product; (iii) a prescription drug product; and/or (iv) an over-the-counter drug product. (v) a vitamin and/or supplement administered as the primary therapeutic agent for a defined therapeutic purpose. The therapeutic purpose excludes mutagen risk reduction.

“PMM” or “Probiotic Medicine Mixture” refers to a configurable composition comprising (i) one or more therapeutic agents and (ii) one or more adjunct components selected to reduce, attenuate, neutralize, or otherwise mitigate the formation, presence, or biological impact of mutagens and/or mutagenic impurities associated with the therapeutic agent(s), including mutagens arising from manufacturing, storage, handling, preparation, administration, digestion, and/or metabolism. This definition is provided by way of example and is not intended to limit the scope of the invention or the claims.

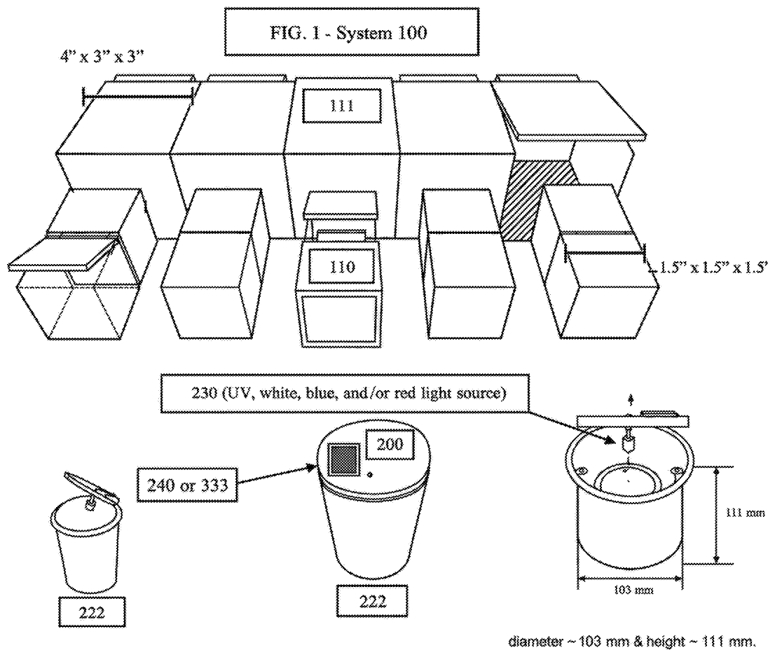

“PMD” or “Probiotic Medicine Dosing System” refers to the dosing and/or preparation system comprising a fermenter and/or an organizer and configured to facilitate the necessary fermentation of the PMM which comprises a therapeutic agent and one or more adjunct components. In some embodiments, the fermenter includes one or more temperature control mechanisms, a temperature monitor, and/or a light source configured to emit one or more wavelengths. This definition is provided by way of example and is not intended to limit the scope of the invention or the claims. (PMD illustrated in FIG. 1 and described herein)

“Mutagen” or “mutagenic species” includes any chemical entity capable of, or potentially capable of, increasing the frequency of genetic mutations or covalent adduct formation in vivo, including without limitation nitrosamines (e.g., N-nitrosodimethylamine (NDMA), N-nitrosodiethylamine (NDEA), nitrosamine drug substance-related impurities (NDSRIs), and related N-nitroso compounds), and other mutagenic reactive intermediates and electrophilic species (including, in certain embodiments, reactive carbonyls and reactive oxygen/nitrogen species) that can contribute to DNA covalent bonding and/or protein covalent bonding.

“Adductomic” and “adductomics” refer to measurement, profiling, and/or assessment (including targeted and/or untargeted workflows) of covalent adducts, including DNA adducts and/or protein adducts, and related biomarkers associated with electrophile burden, oxidative/nitrosative stress, and/or exposure to mutagens and reactive intermediates. Adductomics may be performed as a single timepoint measurement or longitudinally over multiple timepoints.

“Adductomic endpoint” refers to a measurable output derived from adductomics. Non-limiting examples include the abundance of a specific adduct, a class of adducts, and/or a “total adduct burden.”

“Total adduct burden” refers to an aggregate measure of abundance of one or more adduct species and/or adduct classes in a biospecimen (e.g., serum/plasma albumin, hemoglobin, and/or DNA), measured using one or more analytical workflows including LC-MS, LC-MS/MS, LC-HRMS, and/or LC-HRMS/MS, and may be targeted or untargeted.

“Albumin adduct burden” or “total albumin adduct burden” refers to an aggregate measure of abundance of one or more adduct species and/or adduct classes measured on serum albumin in a biospecimen using one or more targeted and/or untargeted adductomic workflows.

“Human serum albumin (HSA) adduct burden” refers to albumin adduct burden measured on human serum albumin.

In human embodiments, HSA adduct burden and total albumin adduct burden refer to the same metric. In veterinary or other non-human embodiments, the analogous metric is the corresponding species-specific serum albumin adduct burden, unless expressly stated otherwise.

“Adjunct,” “adjunct component,” “Component 1,” “Component 2,” “Component 3,” and “Component 4” refer to any agent, supplement, organism, metabolite, excipient, nutrient, vitamin, or additive that is combined with, co-administered with, and/or used to ferment a composition comprising one or more therapeutic agents (e.g., “Component 5”), at or near the point of preparation, dispensing, use, and/or administration. Adjunct components include, without limitation, probiotics and/or other microorganisms (viable or non-viable), coenzymes, scavengers, inhibitors, buffers, antioxidants, chelators, stabilizers, nutrients, vitamins, supplements, foods, and combinations thereof.

“Algae-derived material” refers to material derived from algae and includes, without limitation, whole algae organism, tissue, material, powder, slurry, extract, fraction, tissue, biomatter, purified constituent, and mixtures thereof.

“Comparator data” refers to data used for comparison to biomarker data. Suitable comparator data include, without limitation: (i) administration of the therapeutic agent in the absence of the adjunct component(s); (ii) an otherwise identical composition lacking the adjunct component(s) and/or lacking conditioning/fermentation; (iii) a subject-specific baseline measured prior to administration; (iv) a matched cohort control; (v) a population reference range; and/or (vi) a threshold value.

“Conditioning” refers to fermenting a composition under controlled conditions (e.g., one or more of duration, temperature, pH, light exposure, concentration, and/or order of addition), using fermenting microorganisms and/or enzymes, prior to administration.

“Configured to” refers to designed, arranged, operably coupled, programmed, and/or otherwise set up to perform the recited function. The term does not require that the function be performed in all instances.

“Fermenting” and “fermentation” refer to contacting a composition with microorganisms and/or enzymes under conditions that produce biochemical transformation (including without limitation acidification, redox change, and/or metabolite production), optionally within defined inoculum ranges and time/temperature/pH windows. In some embodiments, fermentation parameters include one or more of duration, temperature, pH, light exposure, concentration, oxygen level, substrate availability, and order of addition.

“Inhibitor” refers to a component that decreases formation and/or activation of a mutagen or reactive intermediate by obstructing a chemical pathway, enzymatic pathway, microbial pathway, and/or other contributing mechanism.

“Microbial-derived material” refers to material derived from microorganisms and includes, without limitation, viable cells, non-viable cells, lysates, cell wall fractions, metabolites, fermentation products, extracts, and mixtures thereof.

“Near the point of use” refers to preparation, dispensing, and/or handling at or near the time and site of administration and/or absorption. For oral administration, the site of absorption includes the stomach and/or gastrointestinal tract; for topical administration, the site includes the skin and/or mucosal surfaces (including nasal, otic, and pulmonary sites).

“Pharmacist” refers to a pharmacist licensed to practice pharmacy under applicable law.

“Component 5” refers to a therapeutic agent and is fermented ex vivo, outside the body, and prior to administration to the subject.

“Plant-derived material” refers to material derived from plants and includes, without limitation, whole plant material, powder, slurry, extract, fraction, plant tissue, biomatter, purified constituent, and mixtures thereof. Where a plant-derived material is provided as an extract, fraction, or purified constituent, an “equivalent amount” refers to an amount providing a comparable level of one or more marker constituents associated with the plant-derived material, optionally selected from total polyphenols, total flavonoids, total anthocyanins, total glucosinolates and/or isothiocyanates, total organosulfur compounds, total tocopherols, total phytosterols, and/or total sulfated polysaccharides.

“Provider” refers to a healthcare professional authorized to provide care and/or direct therapy under applicable law, including, where applicable, physicians and veterinarians and other licensed clinicians.

“Scavenger” refers to a component capable of reacting with, binding, sequestering, and/or otherwise deactivating a mutagen or reactive intermediate to diminish its effective reactivity, bioavailability, or availability.

“Reduce,” “reduces,” “reducing,” and “reduction” refer to a potential decrease in formation, presence, bioavailability, and/or biological activity of at least one mutagenic species and/or at least one mutagen-related adductomic endpoint relative to a comparator. The reduction may be assessed by one or more analytical methods, including measurement of nitrosamines or nitrosation precursors (e.g., nitrite and/or nitrate) and/or biomarker-based monitoring such as adductomics. By way of non-limiting example, reduction may be evidenced by a decrease in one or more protein adduct metrics, including a human serum albumin (HSA) adduct burden, toward a target value (e.g., about 0.1 pmol adduct per mg HSA), as determined by an adductomics assay.

“Functional protein metric”, abbreviated herein as FPM, refers to a directly measured or calculated parameter reflecting protein-associated physiologic performance, assessed according to a predefined assay, biospecimen, time interval, formula, units, baseline, and/or comparator, and decision rule. Non-limiting examples include: (i) hemoglobin oxygen affinity (P50), measured by a specified oxygen-dissociation assay and reported in mm Hg; (ii) INR; (iii) whole-blood clotting time (WBCT), reported in seconds or minutes; (iv) a predefined relationship between change in ferritin and/or transferrin and change in hemoglobin over a defined interval, calculated according to a specified formula; (v) discordance between HbA1c-derived estimated average glucose and a longitudinal average of directly measured blood glucose values over a defined interval, calculated according to a predefined error metric; and (iv) the change in Vitamin B12/Intrinsic Factor Antibody and other FPM biomarkers described herein.

“Tolerability endpoint” refers to a directly measured or recorded indicator of a subject's ability to continue therapy at an intended regimen without clinically meaningful intolerance, assessed according to a predefined scale, interval, baseline, and/or comparator, clinical judgement, and decision rule. Non-limiting examples include death, hospitalization, adverse-event incidence, adverse-event severity grade, dose interruption, dose reduction, discontinuation, local irritation, gastrointestinal intolerance, sedation, dizziness, orthostatic symptoms, palatability-related nonadherence, and combinations thereof.

“Adverse Drug Event” or, ADE, is any unanticipated, untoward, or inopportune medical occurrence associated with the use of a drug in humans, whether or not it is considered related to the drug product. It can also be defined as harm from the drug itself at a normal dose.

### 8.2 Role-Based Classification of Vitamins and Supplements

A vitamin, supplement, or similar material is classified according to its role in the particular embodiment. When administered as the primary intended therapeutic agent, it is classified as Component 5. When included to inhibit nitrosation, scavenge reactive intermediates, buffer local conditions, or otherwise reduce mutagen risk associated with Component 5, it is classified as an adjunct component. Unless expressly stated otherwise, the same material is not counted as both Component 5 and an adjunct component in the same embodiment. In other words, Component 1 comprises one or more vitamins and/or supplements selected specifically to reduce mutagen risk associated with Component 5.

### 8.3 Administration Context Definitions

As used herein, “dietary factors” refers to meal timing, meal composition, nitrate- and/or nitrite-containing foods, and other food-related variables that may affect tolerability, GI nitrosation conditions, compatibility, administration timing, and patients ‘dietary choices, for example: ketogenic diet, vegan, vegetarian, Western, and similar diets.

Unless expressly stated otherwise, all terms are intended to be construed in an open-ended manner, and the singular includes the plural and vice versa.

### 8.4 DETAILED DESCRIPTION OF EXEMPLARY EMBODIMENTS

The disclosed embodiments and summaries are provided for purposes of illustration and are not intended to limit the scope of the present disclosure. Variations, modifications, substitutions, and combinations of the described embodiments will be apparent to those of ordinary skill in the art and are intended to be included within the scope of the present disclosure and the appended claims.

The skilled artisan will understand that the current state of the art and references related thereto are hereby incorporated by reference: For example, AHFS Drug Information (published by ASHP) describes a host of pharmaceuticals approved by the FDA and available for use herein; other references include Physician Desk Reference PDR.net, United States Pharmacopeia—National Formulary (USP-NF), Martindale: The Complete Drug Reference, Remington: The Science and Practice of Pharmacy, Pharmaceutical Formulation: The Science and Technology of Dosage Forms, FDA National Drug Code (NDC) Directory, FDA Orange Book, Drugs@FDA, and many others are known and available to the skilled artisan.

### 8.4.a Overview of Embodiments

In various embodiments, the present disclosure provides pharmaceutical compositions, systems, and methods configured to reduce or mitigate exposure to mutagenic compounds, including nitrosamines and other mutagenic impurities and/or reactive intermediates, during or after administration of one or more therapeutic agents. The therapeutic agent may comprise an active pharmaceutical ingredient (API), a final or marketed drug product, a prescription drug product, an over-the-counter drug product, and, in certain embodiments, a vitamin and/or supplement administered as the primary therapeutic agent for a defined therapeutic purpose. The disclosed embodiments may be applied across a wide range of therapeutic categories, including, without limitation, cardiovascular agents, analgesics, antimicrobial agents, endocrine therapies, drugs associated with one or more nitrosamine drug substance-related impurities (NDSRIs), and central nervous system medications.

In certain embodiments, Component 5 is fermented ex vivo, outside the body, and prior to administration; one or more fermented adjunct components are selected for mutagen mitigation, compatibility, tolerability, and/or adherence; and the regimen is maintained or adjusted based on adductomic endpoints and, in some embodiments, one or more functional protein metrics, tolerability endpoints, adverse drug events (ADEs), and/or other clinical data.

In certain embodiments, the PMM and/or PMD is configured not only to reduce mutagen-related adduct burden, but also to improve tolerability of Component 5, as assessed by one or more predefined tolerability endpoints relative to a comparator regimen while maintaining therapeutic benefit. Non-limiting tolerability endpoints include incidence of treatment discontinuation, dose interruption, severity of gastrointestinal intolerance, local irritation, palatability-related nonadherence, and/or one or more other adverse events assessed according to a predefined scale, time interval, and decision rule.

### 8.4.1 MTM/CMM Integration Embodiments and Tolerability

### 8.4.1 (a) MTM/CMM Integration Embodiments.

In some embodiments, the system interface and/or communication protocol is configured to exchange data and recommendations with a medication therapy management (MTM) module and/or a comprehensive medication management (CMM) module. The MTM/CMM module may comprise a care-management component of a pharmacy information system, payer platform, clinic platform, and/or an electronic health record (EHR) system, and may be configured to manage medication therapy interventions, documentation, adherence activities, and care plans.

### 8.4.1 (b) Adductomics Interface-to-Module Communication.

In some embodiments, the interface is configured to exchange data with a medication therapy management (MTM) module and/or a comprehensive medication management (CMM) module, including transmitting updated preparation instructions for fermenting a composition comprising a therapeutic agent and at least one adjunct component, and/or transmitting regimen recommendations for presentation to a pharmacist, clinician, caregiver, and/or subject.

In one embodiment, in response to receiving biomarker data comprising one or more adductomic endpoints, FPM and tolerability assessments, the MTM/CMM module is configured to generate, store, transmit, and/or display one or more recommendations, prompts, tasks, and/or care-plan updates, including without limitation: (i) patient counseling prompts; (ii) monitoring prompts; (iii) adherence reminders; (iv) scheduling of follow-up biomarker collection and/or adductomics testing; (v) documentation tasks; and (vi) regimen updates.

In some embodiments, the regimen updates specify at least one of: (a) adjunct selection; (b) adjunct amount; (c) timing of adjunct administration relative to administration of the therapeutic agent; (d) therapeutic agent amount; and/or (e) one or more preparation parameters, including one or more fermentation parameters.

In some embodiments, the biomarker and/or adductomic data is processed in combination with clinical data, tolerability, and FPM to generate the updated preparation instructions and/or the regimen recommendations, wherein the clinical data comprises at least one of medication regimen data, diagnosis data, laboratory data, allergy data, dietary data, and/or lifestyle data.

In some embodiments, the clinical data further comprise one or more tolerability endpoints and/or safety endpoints. New or worsening tolerability or safety findings may trigger one or more generated prompts, tasks, and/or care-plan updates, including adverse-event assessment, dose-interruption review, palatability assessment, tolerability-guided counseling, modification of one or more fermentation parameters, redesign of one or more PMM parameters, and/or reevaluation of one or more pharmacokinetic and/or pharmacodynamic considerations.

### 8.4.2 Non-Limiting MTM/CMM Example (Prophetic).

In one embodiment, a subject is administered a therapeutic agent in combination with one or more adjunct components. In a representative prophetic implementation, the therapeutic agent comprises duloxetine (e.g., 60 mg) and the adjunct components comprise a plant-derived Component 2 (e.g., kiwi) and a microbial Component 3 (e.g., Saccharomyces yeast), wherein the therapeutic agent is fermented together with at least one adjunct component to form an aqueous fermented mixture (e.g., a slurry) prior to administration.

Biomarker data focusing on untargeted adductomics endpoints is obtained from a blood sample and processed to determine a total albumin adduct burden by LC-HRMS. Comparator data comprises baseline biomarker data for the subject obtained prior to initiating administration. If, after a defined interval (e.g., 35 days), the total albumin adduct burden has decreased by less than about 13% relative to baseline, a subsequent administration is adjusted by increasing a fermentation duration for the fermented mixture (e.g., increasing from about 2 minutes to about 6 minutes) and by increasing an amount of the microbial adjunct (e.g., doubling the probiotic yeast amount and/or CFU-equivalent level), and the updated preparation instructions are transmitted to the MTM/CMM module. If the total albumin adduct burden decreases by at least about 24% relative to baseline, the fermentation duration is maintained and/or an amount of at least one adjunct component is maintained or reduced in a maintenance phase.

Non-limiting MTM/CMM outputs generated from the foregoing embodiment include:
(i) Communication prompts and assessments with the following:
   (A) Prompt contacting an MTM and/or CMM provider
   (B) Prompt counseling of the subject
   (C) Recommend and/or schedule repeat biomarker collection (including adductomics endpoints)
   (D) Review relevant clinical context to determine an adjustment intensity expected to provide benefit while maintaining acceptable subtherapeutic risk, wherein the clinical context includes, without limitation:
      1. Diagnosis history
      2. Symptom scale scores and clinical monitoring (e.g., PHQ-9, A1C where available)
      3. Concomitant medications
      4. Family and medical history
(ii) Tasks as described below:
   (A) Review clinical data and subject history
   (B) Generate recommendations for the complete patient care team
   (C) Adjust adjunct amount(s)
   (D) Update preparation parameters, including without limitation:
      1. Fermentation duration; and/or
      2. Microbial adjunct concentration setpoints
   (E) Document the intervention within the MTM/CMM module
(iii) Patient care-plan updates as follows:
   (A) Update regimen timing and/or regimen composition
   (B) Set a monitoring interval for:
      1. Clinical and tolerability endpoints
      2. Adductomics endpoints
   (C) Update a goal threshold and/or goal trend for:
      3. Total adduct burden
      4. Total albumin adduct burden
wherein the goal trend optionally comprises achieving a decreasing adductomic trend of at least about 24% relative to baseline.
   (D) Optionally obtain electronic sign-off by one or more care-team members.
      8.4.2 (a) PMM Integration with Clinical Workflow

In some embodiments, the adjunct compositions, preparation parameters, and regimen instructions are integrated into one or more clinical workflow systems, including without limitation electronic prescribing (eRx) systems, pharmacy dispensing systems, MTM/CMM platforms, and/or electronic health record (EHR) systems. In certain embodiments, the clinical system presents one or more preconfigured adjunct options associated with (i) a prescribed therapeutic agent and (ii) a subject profile, and enables selection and/or modification by a prescriber and/or pharmacist based on patient-specific factors including, without limitation, allergies, diagnoses, laboratory values, concomitant medications, dietary factors, and/or prior biomarker or adductomics results.

In some embodiments, the prescribing and/or dispensing system automatically generates and/or recommends an adjunct administration schedule coordinated with administration of the therapeutic agent, including timing relative to dose administration and/or meals, and where applicable timing relative to a defined preparation and/or fermentation window, thereby promoting consistent execution for mutagenic mitigation while maintaining therapeutic efficacy. In certain embodiments, the system further generates prompts and/or tasks for counseling, documentation, and scheduling of follow-up monitoring, including repeat biomarker collection and/or adductomics assessment, tolerability, FPM, analysis for adverse event risks and/or occurrence. In some embodiments, the prescribing/dispensing system may recommend fermentation timing, adjunct selection, or PMM component and preparation changes to improve tolerability, reduce adverse event risks from Component 5 while maintaining therapeutic benefit.

In some embodiments, pharmacies provide the adjunct compositions alongside the primary medication, including as a kit, co-pack, synchronized unit-dose packaging, or other coordinated delivery format, and supply clear patient-facing instructions and educational materials in printed and/or electronic form to improve patient administration, understanding, and compliance.

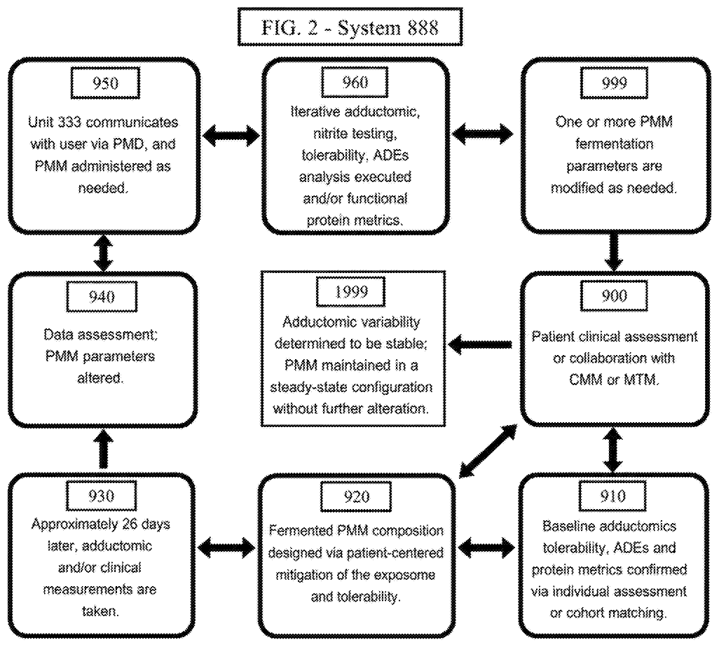

### 8.4.3 PMD System Embodiment and Accompanying FIGS. 1 and 2

In one system embodiment illustrated in FIG. 1, a Probiotic Medicine Dosing (PMD) system, identified as System 100, comprises an organizer and a fermentation vessel. The organizer includes one or more removable modules 110 and/or 111 (e.g., cartridges, compartments, pods, or blocks) configured to store, contain, dispense, and/or present one or more components for preparation of a composition configured for fermentation, including, without limitation, one or more of Components 1-5. In some embodiments, the modules 110 and/or 111 are detachably coupled to the organizer and/or to one another to permit separate storage (e.g., refrigerated storage and/or room-temperature storage) and subsequent reattachment, thereby promoting convenience, dosing flexibility, and adherence as determined by the patient-care team. By way of non-limiting example consistent with FIG. 1, one or more modules 111 may have external dimensions of about 4 in×3 in×3 in, and one or more modules 110 may have external dimensions of about 1.5 in×1.5 in×1.5 in, although other sizes and geometries may be used.

A fermentation vessel 222 defines a chamber configured to ferment a composition comprising a therapeutic agent and at least one adjunct component. The vessel 222 may be provided with a lid 200 configured to close the vessel during fermentation. In some embodiments, the vessel and/or lid includes or supports one or more functional elements, including, without limitation, a light source 230 configured to emit one or more wavelengths selected from ultraviolet, white, blue, and/or red light, and a temperature monitor 240 and/or an interface or communication module 333. In certain embodiments, the temperature monitor 240 and/or interface module 333 further includes one or more sensors (e.g., a temperature sensor, pH sensor, and/or other environmental sensor) and one or more controllers and/or interfaces configured to monitor and/or control at least one preparation and/or fermentation parameter selected from duration, temperature, pH, light exposure, wavelength, concentration, and/or order of addition. By further non-limiting example, fermentation vessel 222 may have an outer diameter of about 103 mm and a height of about 111 mm.

FIG. 2 depicts an exemplary PMM/PMD decision workflow and feedback loop, identified as System 888, for configuring a PMM composition and adjusting PMM and/or PMD preparation parameters based on biomarker data, tolerability data, adverse drug event (ADE) data, functional protein metrics, and/or other clinical data. In some embodiments, System 100 includes an interface and/or communication module 333 configured to receive subject-specific data, wherein the data comprises one or more adductomic endpoints and, in some embodiments, one or more nitrite measurements, tolerability endpoints, ADE data, clinical measurements, and/or one or more functional protein metrics, and to output updated preparation instructions based on the received data. The updated preparation instructions may specify at least one preparation and/or fermentation parameter, including, without limitation, updated fermentation duration, temperature setpoint, pH setpoint, light-exposure constraint, wavelength selection, concentration, administration timing, adjunct amount, and/or order of addition. The updated preparation instructions may be output via the PMD, the interface 333, a user device, printed matter, an electronic message, an audio message, a website, and/or storage on a non-transitory computer-readable medium.

In one embodiment, FIG. 2 illustrates an iterative, closed-loop feedback process. The workflow may begin at step 900, in which patient clinical assessment is obtained, optionally in collaboration with comprehensive medication management (CMM) and/or medication therapy management (MTM). At step 910, baseline adductomics, tolerability, ADEs, and protein metrics are confirmed via individual measurement and/or cohort matching. At step 920, a fermented PMM composition is designed via patient-centered mitigation of the exposome and tolerability, and the PMM regimen is then administered. At step 930, after an exemplary interval of therapy, about 26 days, one or more adductomic and/or clinical measurements are obtained. At step 940, the resulting data are assessed and one or more PMM parameters may be altered. At step 950, unit 333 communicates with the user via the PMD, and the PMM is administered as needed. At step 960, iterative adductomic and/or nitrite testing, tolerability assessment, ADE analysis, and/or assessment of one or more functional protein metrics is executed to further evaluate response and guide optimization. At step 999, one or more PMD and/or PMM parameters are altered as needed. In some embodiments, the workflow may return to one or more earlier steps, including step 900, thereby providing a closed-loop, patient-centered system and method integrating PMD operation, PMM formulation and administration, adductomics, tolerability, ADE monitoring, functional protein metrics, and CMM/MTM collaboration. In some embodiments, the workflow advances to step 1999, in which adductomics and tolerability are stable and the PMM is set to steady state and not altered.

In one non-limiting example consistent with the workflow of FIG. 2 and the use of functional protein metrics, a subject may undergo HbA1c testing and direct blood-glucose monitoring during a corresponding time period, including post-prandial measurements. In this example, the HbA1c result may correspond to an estimated average glucose of about 176 mg/dL, while the subject's directly measured post-prandial blood-glucose values during the same period may yield a numerical average of about 202 mg/dL. The difference, bias, ratio, and/or averaged deviation between the HbA1c-derived estimate and the longitudinal average of directly measured blood-glucose values may be calculated as one or more error metrics constituting an FPM and evaluated in corroboration of the subject's adductomic profile and response, including hemoglobin and/or HSA adduct burden. In some embodiments, a decrease in hemoglobin adduct burden is associated with a decreased error metric. The PMM intervention may then be adjusted, for example by modifying PMD fermentation time and/or one or more operating, preparation, or composition parameters, to reduce one or more hemoglobin adduct concentrations toward a target level. Upon re-testing, a change in the error metric may be used as an objective indicator that the PMM intervention is being adjusted in a clinically appropriate manner. By way of further non-limiting example, similar corroboration may additionally or alternatively be performed using one or more functional protein metrics, including hemoglobin oxygen affinity (P50), INR, whole-blood clotting time (WBCT), a ratio, slope, trend, and/or other relationship between change in ferritin and/or transferrin and change in hemoglobin over a defined interval, and/or other calculated or directly measured biochemical indices reflecting protein function, protein-associated physiologic performance, and/or protein modification, as determined by the patient-care team.

### 8.4.4 Composition Components of PMM and Monitoring

### 8.4.4.1 Therapeutic Agent Embodiments: API, Drug Product, and Therapeutic Supplement

In some embodiments, the therapeutic agent is configured for oral or topical administration.

As used herein, “API” means active pharmaceutical ingredient.

As used herein, “drug product” means a finished dosage form comprising an API and one or more pharmaceutically acceptable excipients.

In some embodiments, the therapeutic agent comprises an API. In certain embodiments, the API is provided as a free base, pharmaceutically acceptable salt, ester, prodrug, solvate, hydrate, polymorph, co-crystal, or other pharmaceutically acceptable form. In other embodiments, the therapeutic agent comprises a final or marketed drug product.

In certain embodiments, the drug product comprises a prescription drug product, an over-the-counter drug product, or a supplement. The drug product is administered as the primary therapeutic agent for a defined therapeutic purpose.

Non-limiting therapeutic categories include analgesics; antihypertensive agents; anti-diabetic agents; anti-depressants and anxiolytics; antimicrobials; hormonal agents; anti-epileptic agents; anti-psychotics; anti-neoplastic agents; cardiovascular agents; immunosuppressants; sedative-hypnotics; muscle relaxants; anti-platelet and anti-coagulant agents; antihistamines; anti-obesity agents; antiparkinsonian agents; anti-dementia agents; and drugs associated with actual or potential nitrosamine drug substance-related impurity (NDSRI) concern.

Representative drugs and drug products within the foregoing categories may be identified by a person of ordinary skill in the art using standard pharmaceutical references and drug-information sources, including AHFS Drug Information, the Handbook of Nonprescription Drugs, Remington: The Science and Practice of Pharmacy, the USP-NF, the FDA National Drug Code Directory, and the Orange Book (Approved Drug Products with Therapeutic Equivalence Evaluations), each as relevant.

In some embodiments, the therapeutic agent comprises a vitamin and/or supplement administered as the primary therapeutic agent for a defined therapeutic purpose, wherein reduction of mutagen risk, standing alone, does not constitute the defined therapeutic purpose. In these embodiments, the therapeutic vitamin and/or supplement is classified as Component 5. Non-limiting examples of such vitamins and/or supplements include vitamins; minerals; herbs and botanicals; amino acids; dietary substances used to supplement the diet; concentrates; metabolites; constituents; extracts; and combinations thereof. In certain embodiments, the vitamin and/or supplement is provided in a capsule, tablet, powder, liquid, softgel, gel, suspension, sachet, or other suitable dosage form for oral or topical administration.

### 8.4.4.2 Adjunct Components

In certain embodiments, one or more adjunct components are selected and configured to reduce formation, concentration, bioavailability, and/or biological activity of mutagenic species (including nitrosamines and other mutagenic impurities and/or reactive intermediates). Non-limiting examples of adjunct components include:
1. Vitamins and/or supplements (Component 1). Representative examples include vitamin C and vitamin E. In certain embodiments, such antioxidants may inhibit nitrosation reactions and/or scavenge electrophilic intermediates, including nitrosating species and related reactive nitrogen intermediates.
2. GRAS plant/algae substances (Component 2). Plant- or algae-derived materials and foods, including plants from the orders Ericales, Rosales, Brassicales, Poales, Asparagales, Cucurbitales, Caryophyllales, Asterales, Lamiales, Malpighiales, Fagales, Fabales, Laurales, and/or Pinales may be used. In some embodiments, such plant-derived materials provide nucleophilic and/or antioxidant constituents that can bind, neutralize, or otherwise attenuate mutagens and/or reactive intermediates. Lastly, in certain embodiments, the brown algae (e.g., class Phaeophyceae, phylum Ochrophyta), red algae (phylum Rhodophyta), and green algae (e.g., phyla Chlorophyta and/or Charophyta) may be used as a component 2. In certain embodiments, Component 2 comprises (i) a whole algae or plant-derived material or food, (ii) a milled powder, (iii) an aqueous slurry, (iv) a standardized extract, or (v) a purified constituent, (vi) plant/algae tissue or bio-matter. In certain embodiments, Component 2 is selected based on the presence of literature data, provider, patient, pharmacist preference, and the presence of bio-nucleophilic and/or antioxidant constituents capable of reacting with nitrosation precursors and/or electrophilic mutagens, including polyphenols, thiols, amines, indoles, carotenoids, chlorophyll derivatives, phycobiliproteins, and enzymes selected from the following non-limiting examples and combinations thereof. Non-limiting representative species and examples include:
Ericales: including blueberries, kiwi fruit, persimmon, huckleberry, and Brazil nuts (anthocyanins/quercetin/selenium)
Brassicales: including broccoli, kale, arugula, Brussels sprouts, cauliflower, collard greens, and mustard greens (glucosinolates/thiols/indoles)
Rosales: including blackberries, hemp seeds, almonds, strawberries, raspberries, and mulberries (polyphenols/tannins)
Poales: including oat, millet, bulgur, fonio, teff, rice, corn, barley, wheat berries, rye berries, sorghum, and pineapple (e.g., providing dietary fibers and phenolic constituents; for pineapple embodiments, proteolytic enzymes such as bromelain).
Asparagales: including onion, garlic, leeks, shallots, scallions, chives, asparagus (allicin/organosulfur compounds/quercetin).
Cucurbitales: including pumpkin seeds, cantaloupe, honeydew, zucchini squash, watermelon (cucurbitacins, phytosterols, squalene).
Caryophyllales: amaranth, quinoa, buckwheat, spinach, rhubarb, Swiss chard (phenolic acids/saponins/betalains).
Asterales: sunflower seed, dandelion greens, chicory endive, artichokes, tarragon (polyphenols/terpenes).
Lamiales: olives, chia seeds, sesame seed, basil, rosemary, thyme, oregano and sage, peppermint (polyphenols/terpenes/kaempferol).
Malpighiales: flax seed, passion fruit, cassava, mangosteen (secoisolariciresinol diglucoside/xanthones/flavonoids).
Fagales: walnuts, pecans, chestnuts, hazelnuts, beechnuts (ellagitannins/tellimagrandin/polyphenols).
Fabales: soybeans, lentils, pinto bean, black beans, chick peas (ellagic acid/anthocyanins, ferulic acid).
Brown algae (e.g., class Phaeophyceae, phylum Ochrophyta): kombu, wakame, green algae (e.g., phyla Chlorophyta and/or Charophyta): aonori, sea grapes; red algae (phylum Rhodophyta): Irish sea moss, nori, ogo, dulse, gim, zicai (e.g., phycobiliproteins and porphyran-type and/or other sulfated polysaccharides, including phycoerythrin-related proteins).
Pinales (conifer-derived materials): pine nuts and/or pine seed material (e.g., Pinus spp., including Pinus pinea), pine pollen (e.g., Pinus pinea pollen), and optionally conifer shoot material (e.g., spruce tips). (pinolenic acid, phytosterols (e.g., β-sitosterol), and tocopherols).
Laurales: avocado, cinnamon, bay leaves (tocopherols, lutein, zeaxanthin, procyanidins, eugenol).
These are non-limiting examples; additional species within the listed order are contemplated.
3. Probiotics and microbial adjuncts (Component 3). In certain embodiments, probiotics include yeasts of the order Saccharomycesles and/or bacteria from the class Actinobacteria, Bacilli, and/or phylum Cyanobacteria. Probiotics may support gastrointestinal microbial balance and, in certain embodiments, may metabolize, sequester, or otherwise reduce exposure to mutagens. In certain embodiments, Component 3 comprises a microorganism selected from yeasts, Actinobacteria, Bacilli, and/or Cyanobacteria. Because different microorganisms have different standard growth and handling requirements, fermentation parameters (e.g., oxygen level, temperature, duration, pH, substrate availability, and/or light exposure) may be selected from the disclosed ranges to match the selected microorganism and implementation, using the monitoring and decision rules described herein.
Actinobacteria (examples): Bifidobacterium spp. (e.g., B. longum, B. breve, B. bifidum)
Bacilli class (examples): Lactobacillus spp. (e.g., L. rhamnosus, L. plantarum, L. acidophilus) and/or Bacillus spp. (e.g., B. coagulans, B. subtilis)
Cyanobacteria (examples): Arthrospira (often sold as “spirulina”), Nostoc, Synechococcus
4. Prebiotic, nutrients, fermentable substrate and/or excipients (Component 4). In certain embodiments, Component 4 comprises one or more fermentable substrates and/or excipients, including sugars such as dextrose, glucose, sucrose, and maltose, which may support probiotic viability and/or modulate fermentation kinetics. In certain embodiments, Component 4 further comprises one or more buffering agents and/or chelators, including bicarbonate, EDTA, and phosphate buffers, among others. In certain embodiments, prebiotics include inulin (e.g., “chicory root fiber”), fructooligosaccharides (FOS) and/or oligofructose, galactooligosaccharides (GOS), and psyllium husk, among others. Without being bound by theory, the adjunct components may act by one or more mechanisms, including scavenging reactive intermediates or mutagens, modifying gastrointestinal permeability, chelating catalytic metals, and/or altering microbial metabolism. In some embodiments, one or more adjunct components are titrated based on patient-specific factors and/or monitoring data to provide an amount effective to inhibit nitrosation and/or scavenge electrophilic species.

In certain embodiments, the total daily amount of adjunct components relative to the therapeutic agent (Component 5) may vary widely. By way of non-limiting example, the API ratio or marketed drug product can be quantified as follows: a ratio of mg API:mg total PMM adjuncts may range from about 1 g: 0.001 mg to about 1 g:1000 g, depending on the potency and dosage form of the therapeutic agent (including microgram-dose products) and the selected adjunct format (including higher-mass plant-derived materials in Component 2).

### 8.4.4.3 Therapeutic Agent (Component 5).

In some embodiments, the therapeutic agent (Component 5) comprises at least one of the following: (i) an active pharmaceutical ingredient (API); (ii) a final or marketed drug product comprising an API; (iii) a prescription drug product; (iv) an over-the-counter drug product; and/or (v) a vitamin and/or supplement administered as the primary therapeutic agent for a defined therapeutic purpose, wherein reduction of mutagen risk, standing alone, does not constitute the defined therapeutic purpose for classification as Component 5.

In certain embodiments, Component 5 is provided in a form suitable for ex vivo mixing and fermentation with at least one adjunct component. For example, a solid dosage form may be comminuted and dispersed in an aqueous medium to form a solution, suspension, or slurry prior to or during fermentation. In other embodiments, Component 5 is provided as a liquid dosage form or as a powder that is directly dissolved or suspended in an aqueous medium for fermentation.

By way of non-limiting example, Component 5 may comprise a subject's prescribed medication, over-the-counter medication, vitamin-based therapy, and/or supplement-based therapy administered as part of the subject's therapeutic regimen.

For clarity, when a vitamin and/or supplement is administered as the primary intended therapeutic agent in a given embodiment, that vitamin and/or supplement is classified as Component 5. By contrast, when a vitamin and/or supplement is included only for mutagen-risk reduction associated with another component, it is classified as Component 1 rather than as Component 5, unless expressly stated otherwise. The same vitamin and/or supplement cannot be classified simultaneously as both Component 1 and Component 5 in the same embodiment.

### 8.4.5 Formulation and Preparation

In certain embodiments, Component 2 comprises a plant-derived material selected in a whole-material, comminuted, slurry, extract, fraction, and/or purified-constituent format. Component 2 may be substituted with another plant-derived material without departing from the disclosure, provided that the substitute is compatible with the subject, Component 5, and the selected ex vivo fermentation regimen. Such substitution may be carried out using conventional processing and formulation operations, including washing, blanching, steaming, drying, milling, grinding, blending, pressing, hydrating to form a slurry, aqueous extraction, alcohol extraction, supercritical extraction, filtration, concentration, fractionation, encapsulation, and combinations thereof.

In certain embodiments, plant-derived materials may be provided in a powder, slurry, beverage, capsule-fill, tablet-excipient blend, suspension, dispersion, or other ingestible format suitable for combination with the PMM.

In certain embodiments, where an extract, fraction, or purified constituent is substituted for a whole material, or vice versa, the substituted material may be provided in an equivalent amount selected to provide a comparable level of one or more marker constituents disclosed herein. In certain embodiments, equivalence is based on one or more predefined marker-constituent measures associated with the selected plant-derived material, as otherwise described herein.

In certain aqueous embodiments, a daily PMM dose is provided as an aqueous suspension and/or dispersion and is stored in an appropriate container under conditions suitable for the selected formulation. In certain embodiments, the formulation is assigned a beyond-use date determined according to applicable stability, microbial, and handling considerations. In some such embodiments, fermentation is initiated by addition of an activated Component 3 and, where applicable, one or more selected Component 4 materials.

In certain non-aqueous embodiments, the PMM is provided as a dry, semi-solid, and/or other non-aqueous formulation configured for ex vivo fermentation following final addition of water and one or more selected adjuncts prior to administration. Non-limiting non-aqueous forms include oral tablets, capsules, powders, sachets, reconstitutable powders, creams, ointments, oils, foams, and patches.

In certain embodiments, the selected formulation format, water content, order of addition, and/or activation conditions are chosen based on compatibility with the therapeutic agent, compatibility with the selected adjunct components, the intended route of administration, and the desired fermentation window.

### 8.4.6 Methods of Configuration and Ex Vivo Preparation

The following methods are illustrative and non-limiting and may be selected, configured, and modified by a provider and/or pharmacist and, in some embodiments, executed by a user or caregiver in accordance with provider- and/or pharmacist-directed instructions.

In certain embodiments, instructions for operating the Probiotic Medicine Dosing System (PMD) and configuring the Probiotic Medicine Mixture (PMM) are determined by a provider and/or pharmacist based on patient-specific factors, the identity and dosage form of the therapeutic agent (Component 5), compatibility constraints, clinical parameters, and analysis of adductomic monitoring and/or other biomarker data, including, in some embodiments, one or more functional protein metrics. The PMM composition and PMD operating parameters may therefore be individualized at the subject level and may be modified over time in response to clinical response and/or monitoring results. In certain embodiments, such configuration includes selecting one or more adjunct components and determining one or more ex vivo fermentation parameters for fermenting the therapeutic agent together with at least one adjunct component outside the body and prior to administration.

In certain embodiments, Component 5 is fermented ex vivo, outside the body, and prior to administration; one or more fermented adjunct components are selected for mutagen mitigation, compatibility, tolerability, and/or adherence; and the regimen is maintained or adjusted based on adductomic endpoints and, in some embodiments, one or more functional protein metrics, tolerability endpoints, adverse drug events (ADEs), and/or other clinical data.

### 8.4.6 (a) Component 2 Selection and Design (Plant-Derived Materials)

In certain embodiments, Component 2 comprises a plant-derived material selected by a provider and/or pharmacist and configured for compatibility with the subject, the therapeutic agent, and the ex vivo fermentation regimen described herein. Non-limiting plant-derived materials include, without limitation, seeds, leaves, florets, stems, roots, fruits, and powders, slurries, extracts, fractions, and/or purified constituents derived therefrom, provided that the selected material is compatible with the subject (e.g., allergy and intolerance constraints), with the therapeutic agent, and with the preparation and administration protocol described herein.

By way of non-limiting examples, Component 2 may comprise chia seed material, flaxseed material, oat fiber, apple pectin, citrus pectin, broccoli and/or other Brassica plant material, leafy greens, berries, and/or dried plant powders and mixtures thereof. In certain embodiments, Component 2 is selected based on one or more considerations including, without limitation, availability, palatability, preparation feasibility, anticipated antioxidant and/or nucleophilic constituent profile, avoidance of clinically meaningful food-drug interaction risk, and compatibility with one or more selected fermentation parameters.

In certain embodiments, solid plant materials (e.g., seeds, florets, leaves, and/or dried powders) are prepared by portioning a provider-selected mass, optionally rinsing and/or trimming as appropriate for food-grade use, and comminuting (e.g., blending and/or milling) the material with potable drinking water to form a slurry, puree, or dispersion suitable for combination with the PMM mixture. In some embodiments, a water-to-solid ratio is selected to achieve a pourable consistency, for example about 1:1 to about 20:1 by mass, although other ratios may be used. The resulting slurry may be introduced into the fermenter prior to initiation of fermentation or during fermentation. In other embodiments, the resulting slurry may be combined with the fermented therapeutic composition after completion of fermentation and before administration, provided that the therapeutic agent has undergone ex vivo fermentation prior to administration.

In certain embodiments, Component 2 is provided in an unsalted and un-pickled form. In further embodiments, fermentation duration is controlled and not excessive, as prolonged fermentation under certain combinations of composition, temperature, and/or subject- or pharmaceutical-specific factors may increase formation and/or bioavailability of undesirable compounds, including mutagenic species, and/or may increase nitrosating conditions.

In one embodiment, one or more PMD preparation parameters—including (a) fermentation duration, (b) fermentation temperature setpoint, (c) pH, (d) light exposure (including wavelength and exposure duration), and/or (e) order of component addition—are selected and, where appropriate, adjusted iteratively within the disclosed ranges based on one or more predefined criteria and/or adductomic monitoring data as described herein

### 8.4.6 (b) Fermentation Duration and Related Parameters

### Fermentation Duration

In certain embodiments, fermentation duration is selected from about 1 minute to 24 hours; in some embodiments from about 5 minutes to 6 hours; in further embodiments from about 10 minutes to 60 minutes; and in certain preferred embodiments from about 2 minutes to 10 minutes.

In other embodiments, including extended-fermentation implementations, fermentation duration is selected from about 1 day to 7 days; in some embodiments about 2 days to 5 days.

In still other embodiments, fermentation duration is selected from about 1 week to 3 weeks; in some embodiments about 1 week to 2 weeks.

The foregoing ranges may be applied to different adjunct selections, microbial selections, and/or objectives described herein, and are provided as non-limiting examples.

### Fermentation Temperature Setpoint

In certain embodiments, the fermentation temperature setpoint is about 15° C. to 45° C.; in some embodiments about 20° C. to 40° C.; and in certain preferred embodiments about 25° C. to 35° C.

In some embodiments, the PMD temperature measurement uncertainty is about ±1° C. (or a comparable tolerance), and temperature ranges and set points are interpreted accordingly.

pH

In certain embodiments, pH of the PMD mixture is within about pH 2.5 to pH 10.5; in some embodiments about pH 2.5 to pH 8; in further embodiments about pH 3.0 to pH 7.5; and in certain preferred embodiments about pH 3.5 to pH 6.5.

In some embodiments, pH is adjusted or maintained using one or more buffers disclosed herein.

### Light Exposure

In certain embodiments, the PMD includes a light source configured to illuminate the mixture at one or more wavelengths. Non-limiting examples include:
white light (broad spectrum);
blue light having a peak wavelength within about 380 nm to 500 nm;
red light having a peak wavelength within about 620 nm to 750 nm; and/or
ultraviolet (UV) light within about 100 nm to 380 nm (optionally including UV-A, UV-B, and/or UV-C sub-bands).

In some embodiments, light exposure duration is about 3 seconds to 60 minutes; in some embodiments about 5 seconds to 10 minutes; and in certain preferred embodiments about 10 seconds to 120 seconds. In some embodiments, light exposure comprises a pulse of about 5 seconds to 100 seconds. In some embodiments, light exposure is omitted.

### Order of Addition

In certain embodiments, the order of component addition comprises: (i) adding one or more of fermented Components 1, 3, and 4 to water and mixing prior to addition of Component 2; (ii) adding Component 2 as a slurry prior to addition of one or more other fermented components; and/or (iii) adding the therapeutic agent (Component 5) with only 1-5 seconds of fermentation time and subsequently administering the resulting composition.

### 8.4.6 (c) Selection Rules on Administration Timing.

In certain embodiments, the PMM mixture is administered prior to, substantially concurrently with, or subsequent to administration of the fermented therapeutic agent (Component 5), wherein the selected timing is based on at least one of: the identity and dosage form of Component 5, patient-specific factors (e.g., age, organ function, comorbidities, concomitant medications), and the identity and mechanism of the selected adjunct component(s). By way of non-limiting example, the fermented PMM mixture is administered from about 0 to 120 minutes before the fermented Component 5, concurrently with the fermented Component 5, or from about 0 to 240 minutes after the fermented Component 5.

In some embodiments, the PMM regimen includes a loading phase comprising an increased administration frequency and/or increased adjunct amount for an initial interval, followed by a maintenance phase comprising a reduced administration frequency and/or reduced adjunct amount. In certain embodiments, a transition between the loading phase and the maintenance phase is determined based on monitoring data, including without limitation clinical response, tolerability, and/or biomarker data comprising adductomics endpoints.

In certain embodiments, subsequent timing, sequencing, and/or frequency of administration are adjusted not only according to adductomic response, but also according to one or more tolerability endpoints, including without limitation any adverse event that may be related to Component 5, gastrointestinal intolerance, palatability-related nonadherence, local irritation, sedation, dizziness, and/or dose-interruption history.

In certain embodiments, sequencing and timing are selected to account for food effects, including administration relative to meals and/or ingestion of nitrate- and/or nitrite-containing foods, such that conditions that promote nitrosation are reduced while maintaining therapeutic efficacy of the fermented component 5. In some embodiments, administration timing is selected to reduce gastric or intestinal conditions associated with nitrosation and/or electrophile formation.

In other embodiments, administration of at least one fermented adjunct component is independent of administration of fermented Component 5, including without limitation administration as a separate dosage form at a different time of day (e.g., morning or evening), on different days of the week, and/or according to a periodic schedule (e.g., weekly or monthly), optionally based on monitoring data and/or patient adherence considerations.

### 8.4.7 Adductomics

In certain embodiments, Component 5 is fermented ex vivo, outside the body, and prior to administration; one or more fermented adjunct components are selected for mutagen mitigation, compatibility, tolerability, and/or adherence; and the regimen is maintained or adjusted based on adductomic endpoints and, in some embodiments, one or more functional protein metrics, tolerability endpoints, adverse drug events (ADEs), and/or other clinical data.

In one embodiment, adductomic data are obtained or extrapolated before initiation of the PMM regimen and iteratively at one or more follow-up intervals to evaluate whether the PMM and/or PMD is associated with reduced mutagen burden relative to baseline and/or comparator data. In certain embodiments, adductomics is performed using a targeted and/or untargeted workflow and one or more analytical platforms selected from LC-MS, LC-MS/MS, LC-HRMS, and/or LC-HRMS/MS.

In some embodiments, the biospecimen is selected from blood, serum, plasma, albumin-containing samples, hemoglobin-containing samples, DNA-containing samples, urine, saliva, stool, buccal cells, local tissue samples, and/or combinations thereof. Non-limiting adductomic endpoints include total adduct burden, total albumin adduct burden, human serum albumin (HSA) adduct burden, hemoglobin adduct burden, DNA adduct burden, methylation-related DNA adduct markers, one or more specific adduct classes, and/or one or more specific adduct species.

In certain embodiments, comparator data comprises one or more of: subject-specific baseline data, matched-cohort data, a population reference range, a threshold value, administration of Component 5 in the absence of one or more adjunct components, and/or administration of a comparable non-fermented composition. In some embodiments, adductomic data are reviewed longitudinally according to a predefined monitoring cadence.

In certain embodiments, the adductomic results are assessed together with one or more functional protein metrics, tolerability endpoints, adverse drug events (ADEs), and/or other clinical data to determine whether PMM composition, component identity, component amount, fermentation parameters, administration timing, and/or monitoring cadence should be maintained or adjusted. In some embodiments, the adductomic results are further reviewed for association with changes in ADE incidence, ADE severity, dose interruption, hospitalization, and/or other clinically relevant outcomes relative to baseline and/or comparator data. Any such association may support continuation, modification, or redesign of the PMM and/or PMD regimen, but no single clinical outcome is required in all embodiments.

In certain embodiments, the PMM and/or PMD is designed not only to reduce mutagen-related adduct burden, but also to reduce the frequency, severity, and/or clinical consequences of one or more ADEs associated with Component 5.

### 8.4.8 Protein Functional Metrics

In some embodiments, functional protein metrics are used to corroborate whether an observed decrease, lack of decrease, or ambiguous change in one or more adductomic endpoints is accompanied by a corresponding change in protein-associated physiologic performance for a given subject or cohort. Baseline comparator data are obtained or estimated, and one or more subsequent follow-up measurements are obtained according to a predefined interval. Unless otherwise specified, A denotes the change between baseline and a designated follow-up interval for the subject.

In some embodiments, using clinical judgment, the patient-care team may exclude, retain, weight, or disregard any PFM subset based on clinical relevance, assay availability, data quality, and/or subject-specific factors. In one non-limiting embodiment, a total PFM corroboration score greater than 4 supports a determination that an observed decrease in adductomic burden is corroborated by one or more functional readouts.

In one embodiment, selected functional protein metrics are combined in a predefined scoring framework described herein and established before review of follow-up data. The scoring framework specifies, for the relevant subject or cohort, the included functional protein metrics, assays, biospecimens, units, formulas, comparators, thresholds, and point assignments. In certain embodiments, one or more metrics may be excluded only according to predefined exclusion criteria specified before follow-up evaluation and based on provider clinical judgement. A total score at or above a predefined threshold may support, but does not by itself always establish, a determination that an observed change in adductomic burden is corroborated by one or more functional readouts.

In one embodiment, non-limiting PFM subsets include the following in a blood biospecimen:

### (a) Hemoglobin Oxygen Affinity (P50).

If |Measured Hb P50-27 mm Hg| is less than 6 mm Hg at baseline and less than 2 mm Hg at one or more subsequent iterations, assign 2 points.

P50 may be measured by tonometry using a Hemox Analyzer and reported in mm Hg.

### (b) Ferritin/Transferrin/Hemoglobin Relationship Metric:

$$If ( Δ ferritin \times Δ transferrin \times mean TSAT ) / ( Δ hemoglobin ) \hat{} 2 < 77 , 500 ,$$

$$assign 1 point .$$

Ferritin is reported in ng/ml, transferrin in mg/dL, TSAT as a predefined percentage or fraction for the selected calculation, and hemoglobin in g/dL.

### (c) Vitamin B12/Intrinsic Factor Antibody Relationship Metric.

If [ΔVitamin B12/Δintrinsic factor antibody]>950, assign 1 point; or if >1600, assign 2 points.

Vitamin B12 is reported in ng/mL. The intrinsic factor antibody is reported in AU/mL; antibody assay type, units, and normalization method are predefined for the selected laboratory workflow.

### (d) Ferritin/Transferrin/Hemoglobin Relationship Metric 2.

$$If [ ( Δ ferritin \times 10 \hat{} - 7 ) / Δ hemoglobin ] + [ ( Δ transferrin \times TSAT \times 0.01 ) / Δ hemoglobin ]$$

falls within a predefined range of 0.85 to 1.55, assign 2 points.

Ferritin is reported in ng/ml, transferrin in mg/dL, TSAT as a predefined percentage or fraction for the selected calculation, and hemoglobin in g/dL.

### (e) INR.

If |INR−0.9| is less than 0.15 at baseline and less than 0.05 after one or more subsequent iterations, assign 1 point.

### (f) Whole-Blood Clotting Time (WBCT).

If |WBCT-280 seconds| is less than 80 seconds at baseline and less than 60 seconds after one or more subsequent iterations, assign 1 point.

WBCT is reported in seconds.

### (g) HbA1c/Direct-Glucose Discordance.

If |calculated average glucose derived from HbA1c-longitudinal average of directly measured blood glucose values| is less than 50 mg/dl at baseline and less than 5 mg/dl after one or more subsequent iterations, assign 2 points.

Blood glucose is reported in mg/dL.

### (h) TSAT.

If |TSAT-45| is greater than 9 at baseline and less than 3 at one or more subsequent iterations, assign 1 point.

TSAT is reported in %.

### (i) if Δ Vit K/Δ Protein C >300 and <900 Assign 1 Point

Vitamin K reported in ng/ml.

### (j) if Δ Nitric Oxide/Mean Erythrocyte Acetylcholinesterase >0.16 and <1.90, Assign 1 Point

Nitric Oxide reported in μmol/L. Erythrocyte acetylcholinesterase reported in activity per Hemoglobin

### 8.4.9 Exemplary Bumetanide Embodiment (Representative Prophetic Configuration)

By way of illustration, and not limitation, a patient-care team comprising a patient, one or more providers, and/or one or more pharmacists will be configuring a PMM mixture and PMD operating parameters for use with bumetanide therapy (e.g., bumetanide 1 mg tablets) as follows:

### PMM Mixture

1. Component 1 (vitamin: ascorbic acid, fine powder, 50 mg
2. Component 2 (GRAS plant): chia seeds (Salvia hispanica), 30 g
3. Component 3 (probiotic): Saccharomyces boulardii, 250 mg
4. Component 4 (nutrient/excipient): sucrose NF, 1 g
5. Component 5 (therapeutic agent): bumetanide tablet, 1 mg (e.g., NDC 69238-1490-1)

### Bumetanide PMD Protocol

The exemplary bumetanide prescription SIG may read as follows:

Take 1 tablet every morning with the PMM and PMD such that your adductomics is less than 0.1 μmol per mg serum albumin. May increase to 3 tablets or decrease to 0.5 tablets as needed to maintain BP of 130/75 mm Hg.

The exemplary bumetanide patient or User may read the PMD instructions as follows:
1. Water. Use FDA- or EPA-approved drinking water.
2. Preparation of chia seeds. Remove the chia-seed component from the PMD and blend with water to form a slurry; set aside.
3. Prepare fermenter. Add warm drinking water to the fill line in the fermenter and confirm a temperature of about 28-33° C.
4. Load components. Remove Components 1 (Vitamin C), Component 3 (the probiotic), Component 4, and bumetanide from the PMD and place them into the fermenter. Close the lid.
5. Fermentation step. Allow fermentation for 3 minutes.
6. Add the blended chia seeds slurry to the fermenter and mix.
7. Administration. Ingest the total mixed contents.
8. Report to the Lab this September for one-time blood draw and monitoring.
   8.4.9 (a) Bumetanide Exemplary Provider PMM Protocol with MTM/CMM (Prophetic and Illustrative Only)

In one representative embodiment, a provider will establish a treatment regimen in which a therapeutically effective amount of bumetanide (Component 5) will be administered in combination with a PMM mixture prepared using selected adjunct components (Components 1-4) and one or more configured PMD operating parameters. By way of non-limiting example, bumetanide will be administered orally at an initial dose of 1 mg once daily, with optional titration up to about 3 mg per day (e.g., about 1-3 mg/day) based on the subject's clinical status, tolerability, and therapeutic response.

In certain embodiments, one or more of (i) the selection and/or amount of the adjunct components and/or (ii) one or more PMD preparation parameters (including conditioning and/or fermentation duration, temperature setpoint, pH, light exposure, and/or order of component addition) are adjusted iteratively within disclosed ranges based on monitoring data. Suitable monitoring data may include analytical measurements of nitrosamines and/or nitrosation precursors (including nitrite and/or nitrate) and/or biomarker-based monitoring such as targeted or untargeted adductomics.

In some embodiments, the monitoring-guided adjustment will be performed using one or more predefined criteria, including a measurable reduction relative to a comparator and/or a trend toward a predefined target range for one or more adductomic endpoints, while maintaining therapeutic benefit of the bumetanide therapy and meeting predefined tolerability and/or compatibility constraints. By way of non-limiting example, a target range may include an HSA adduct burden at or below about 0.1 μmol adduct per mg HSA, or another predefined threshold selected for a given subject and use scenario, without requiring attainment in all subjects. A target clinical goal may be a BP of 130/75 mmHg.

In certain embodiments, the PMM/PMD regimen will be integrated with medication therapy management (MTM) and/or comprehensive medication management (CMM) workflows such that regimen updates, monitoring results, and adjustment recommendations are communicated to the patient-care team using a coordinated communication protocol. In some embodiments, upon generation of updated clinical data by any one of (i) the PMM/PMD workflow (e.g., updated preparation parameters or component selection), (ii) MTM workflow (e.g., adherence assessment, drug-drug interaction screening, dose optimization, or motivational interviewing), or (iii) CMM workflow (e.g., structured care plan updates and follow-up intervals), the protocol further triggers one or more corresponding updates by at least one other workflow within a defined communication window. In this manner, PMM/PMD monitoring and adjustment may be operationally coupled to MTM/CMM interventions to improve continuity of care, adherence, and engagement, including when one aspect of the regimen underperforms or requires modification.

The foregoing dosing amounts, schedules, parameters, monitoring criteria, and communication workflows are provided as representative examples and may be varied by a provider and/or pharmacist according to patient-specific factors and the selected implementation of the PMM and/or PMD.

### 8.4.10 Exemplary Patient-Facing Preparation and Administration Instructions (A Representative Method)

The following procedure is provided for illustrative purposes and may be modified by a provider and/or pharmacist based on the subject, therapeutic agent, and selected components.

The exemplary patient or user may read the PMD instructions as follows:
1. Water. Use FDA- or EPA-approved drinking water.
2. Preparation of Component 2. Remove Component 2 from the PMD and blend with water to form a slurry; set aside.
3. Prepare fermenter. Add warm drinking water to the fill line in the fermenter and confirm a temperature of about 28-33° C.
4. Load components. Remove Components 1, 3, 4, and 5 from the PMD and place into the fermenter. Close the lid.
5. Fermentation step. Allow fermentation for approximately 3 minutes.
6. Add the blended Component 2 slurry to the fermenter and mix.
7 Administration. Ingest the contents according to the provider- and/or pharmacist-directed regimen.

### 8.4.11 Selection Rules, Assay Protocol, Timing, Sequencing, Dose Ranges, and Regimen Phases

In some embodiments, biomarker data is obtained after a defined interval (e.g., 28 days) and compared to the threshold value and/or a subject baseline value. If the HSA adduct burden fails to decrease by at least a predefined amount (e.g., less than about 17% reduction relative to the subject baseline) and/or remains above the interim target, the care team advances to a subsequent iteration by modifying at least one of: (i) Component 2 identity and/or amount, (ii) fermentation duration, temperature, and/or pH, (iii) timing of the fermented adjunct administration relative to fermented Component 5, and/or (iv) therapeutic agent amount. Two exemplary rules include: (I) If burden drops to ≤0.1 μmol/mg HSA, then maintain regimen. Expected result ranges within ≥17% reduction and (II) approaches ≤0.1 μmol/mg HSA while the deviation between the directly measured blood glucose average and A1C is no greater than 6%.

### 8.4.12 Selection Rules on Composition of PMM and its Iterations

In certain embodiments, the PMM mixture baseline for most patients and regimes is as follows:
1. Component 1 (vitamin: ascorbic acid, fine powder, 50 mg) q.s. as needed.
2. Component 2 (GRAS plant): chia seeds (Salvia hispanica), 20 g.
3. Component 2 (GRAS plant): Kiwi (Actinidia deliciosa) 40 g if preferred.
4. Component 2 (GRAS algae): Irish Sea moss 15 g as needed.
5. Component 3 (probiotic): Saccharomyces boulardii, 250 mg.
6. Component 4 (nutrient/excipient): sucrose NF, 1 g.
7. Component 5 (therapeutic agent): As prescribed.
   8.4.12 (a)(i) Initial Selection of Component 2 (Palatability+Feasibility).

In certain embodiments, a first PMM iteration is initiated by selecting Component 2 based on patient palatability preferences and practical preparation constraints.

8.4.12 (a)(ii) Drug-Food Interaction Screen.

Prior to use of the selected Component 2, a pharmacist, provider, or patient-care team member evaluates whether Component 2 presents a clinically significant drug-food interaction with the therapeutic agent (Component 5) and/or the subject's concomitant pharmacotherapy. If a clinically significant interaction is identified, Component 2 is rejected and a different Component 2 is selected.

8.4.12 (a)(iii) Constituent Identification within Component 2.

If Component 2 passes the interaction and patient preference screen, the patient-care team identifies one or more abundant antioxidant and/or scavenger constituents associated with Component 2, including without limitation polyphenols, tannins, flavonoids, thiols, indoles, organosulfur compounds, and combinations thereof.

8.4.12 (a)(iv) Reaction Prediction and/or Kinetic Modeling Against Target Mutagen(s).

In some embodiments, the patient-care team evaluates predicted attenuation of a target mutagenic species associated with Component 5 and/or the subject's lifestyle, diet, concomitant pharmacotherapy, including without limitation an NDSRI and/or another potential mutagen validated by literature review and/or professional judgement, by performing reaction prediction and/or kinetic modeling between (A) the identified antioxidant/scavenger constituents and (B) the target mutagenic species. Modeling may employ, without limitation, reaction outcome prediction, reaction pathway prediction, density functional theory (DFT), kinetic mechanism generation, microkinetic modeling, biochemical network simulation, and/or ODE-based kinetic simulation. Non-limiting tools include COPASI, ORCA, Quantum ESPRESSO, Gaussian, Q-Chem, Molpro, NWChem, CP2K, PHREEQC, Kintecus, and RMG.

8.4.12 (a)(v) Acceptance Criteria for Component 2.

If the modeling indicates that one or more constituents of Component 2 are predicted to chemically react with, quench, trap, or otherwise attenuate the target mutagenic species under relevant conditions (e.g., via a favorable reaction pathway and/or estimated rate constant), the selected Component 2 is accepted for use in the PMM mixture. If not, Component 2 is rejected, and a different Component 2 is selected.

8.4.12 (a)(vi) Biomarker Monitoring and Steady-State Assessment.

Following one or more administrations of the PMM mixture, biomarker data comprising adductomics endpoints is obtained and compared to comparator data. In some embodiments, the comparator data comprises at least one of subject-specific baseline data, a population reference range, or a threshold value. In some embodiments, the patient-care team determines whether an adductomic burden has reached steady state and/or whether a predefined reduction has been achieved relative to comparator data (e.g about a 19% decrease for baseline pmol per mg of HSA untargeted adductomics).

8.4.12 (a)(vii) Iteration Escalation Rules.

If the adductomic burden remains above a threshold (for example, greater than about 0.1 μmol adduct per mg HSA) and/or if a reduction in one or more adductomic endpoints is less than a predefined value (for example, less than about 11% relative to comparator data) after a predetermined number of iterations (e.g., three to five iterations of Component 2 substitution and/or parameter adjustment), the PMM mixture is advanced to a subsequent iteration. This advancement is supported by functional protein metrics which, by way of example, show hemoglobin P50 not trending toward about 25 mm Hg±2 mm Hg. Thus, for subsequent iterations, the patient-care team may modify one or more additional variables, including without limitation substitution of Component 3 (e.g., a fermentation organism), adjustment of fermentation parameters (e.g., duration, temperature, pH, order of addition, and/or light exposure), adjustment of adjunct selection, adjunct amount, and/or timing, and/or adjustment of therapeutic agent amount, until a desired biomarker trend or threshold is achieved.

### 8.4.13 Mechanism of Action

Referring in more detail to the potential mechanism of the PMM and/or PMD: The provider-approved combination of components (1), (2), (3), (4) is formulated to effectively minimize the risk of sub-therapeutic therapy for the patient while maximizing the risk reduction of mutagen exposure. The PMM supplies bio-nucleophiles in an environment of ultra-low-concentration, but potent mutagens allowing for short and effective fermentation times.

In certain embodiments, Component 2 comprises a plant- and/or algae-derived material providing a polyphenolic and bio-nucleophilic sink, including polyphenolic constituents such as flavonoids and/or tannins having multiple hydroxyl (—OH) groups and, in some embodiments, relatively high molecular weight. Without being bound by theory, such hydroxyl-containing polyphenols may provide nucleophilic and redox-active sites capable of (i) reacting with, trapping, and/or sequestering nitrosating species and other electrophilic reactive intermediates, (ii) quenching reactive nitrogen and/or oxygen species, and/or (iii) otherwise inhibiting pathways that promote formation and/or bioavailability of mutagenic species. In certain embodiments, the polyphenolic constituents donate electrons and/or hydrogen equivalents in redox reactions that can reduce nitrite into nitric acid and nitrosamine functional groups into scavenger adducts and benign nitrogen gas.

Nitrosamine formation can be promoted under acidic conditions when nitrosating species derived from nitrite contact amine-containing compounds (e.g., secondary and/or tertiary amines); accordingly, consumption, neutralization, and/or sequestration of nitrite and/or nitrosating species can reduce nitrosamine formation and associated mutagenic risk.

In summary, without being bound by theory, the microbial Component 3 and probiotics may function within the PMM/PMD:
1. Nitrosation inhibition and sequestration
2. Electrophile scavenging
3. Mutagen sequestration and suicidal inhibition
4. Adduct burden reduction and provision of a bio-nucleophilic sink
5. Microbiome enhancement through competitive resistance and metabolic interference.

In summary, without being bound by theory, the compositions may reduce mutagen exposure and the risk of adverse events via: Chemical neutralization of reactive species; Binding or sequestration of mutagenic intermediates; Enzymatic degradation of mutagenic compounds; Modulation of gastrointestinal pH or microbiota; Prevention of precursor formation; Alteration of gastrointestinal mucosal permeability.

In certain embodiments, one or more drug-metabolite measures, including lamotrigine-N-oxide in lamotrigine-related embodiments, are evaluated as potential mechanistic contributors without requiring any particular metabolite to be established as causal for rash or any other adverse event.

### 8.4.14 Patient-Specific Customization

In certain embodiments, the PMM compositions, PMD operating parameters, and associated dosing regimens are individualized for a subject based on patient-specific and/or cohort-specific factors. Such factors may be used to select adjunct components, establish dose ranges, determine fermentation parameters and timing along with sequencing relative to administration of the fermented therapeutic agent (Component 5) and/or meals, and to define loading and maintenance phases and adjustment criteria. Non-limiting customization inputs include:
Subject age and developmental status;
Metabolic profile and physiologic status (including hepatic and renal function);
Gastrointestinal function and microbiome-related factors;
Concomitant medications and potential drug-drug or drug-nutrient or drug-disease interactions;
Dietary patterns and timing relative to meals, including intake of nitrate- and/or nitrite-containing foods;
Medical history and comorbid conditions;
Clinical history and longitudinal response to prior regimens;
Patient preferences and adherence considerations; and
Adductomic monitoring and history
   Pharmacogenomic and/or other molecular or biomarker data.
Functional protein metrics

In some embodiments, one or more of the foregoing factors are incorporated into an iterative adjustment process in which adjunct selection, composition, dose, and/or PMD operating parameters are modified over time to maintain therapeutic benefit while mitigating exposure to mutagenic species.

This enables individualized risk mitigation strategies.

### 8.4.15 Safety and Compatibility

In certain embodiments, the effectiveness biomarkers and endpoints may comprise human serum level of the API or API metabolite and untargeted or targeted adductomics on albumin, hemoglobin, or DNA along with FPM and tolerability.

In certain embodiments, the method, frequency, and choice to collect and analyze standard or innovative untargeted or targeted adductomics are up to the patient-care team, which comprises providers, patients, and/or pharmacists.

In certain embodiments, the patient population comprises the general population, including inpatient and/or outpatient subjects or users, human or animal.

In certain embodiments, where an adjunct component or PMM comprises only food-grade material that is Generally Recognized as Safe (GRAS) for its intended use and is administered within customary dietary intake ranges, and where the therapeutic agent (Component 5) is prescribed and administered in accordance with approved labeling and/or provider direction, co-administration of the adjunct component or PMM with fermented Component 5 is expected to present a low likelihood of introducing additional clinical risk beyond that associated with Component 5 alone, absent a known or clinically meaningful food-drug interaction, allergy or intolerance, or other patient-specific contraindication. In most embodiments, compatibility is further supported by provider and/or pharmacist-directed selection of adjunct identity, amount, and timing relative to fermented Component 5 and meals.

In certain embodiments, compatibility assessment includes evaluation of potential food-drug and drug-drug interactions, contraindications (including allergy or intolerance to plant-derived materials or microbial components), and clinical conditions in which probiotic or fermentable substrates may be used with caution (e.g., immunocompromised status or impaired gastrointestinal barrier function). Nothing herein is intended to require that any particular adjunct component is universally safe or compatible for all subjects or all therapeutic agents; rather, the disclosed embodiments provide a configurable framework in which components and parameters may be selected and adjusted to support safety, tolerability, and compatibility in a given clinical use scenario.

8.4.16 Clinical Interactions with the PMM Fermentation

The disclosed systems, compositions, protocols, and methods may have practical utility in reducing formation, presence, and/or biological impact of nitrosamines and other mutagenic species associated with therapeutic agents, diet, and exposome, while maintaining pharmacotherapeutic benefit. In some embodiments, adjunct components are selected, designed, and configured to avoid clinically meaningful interference with absorption, metabolism, distribution, or excretion of the fermented therapeutic agent (Component 5 or API) and to avoid creating conditions that may increase formation or bioavailability of undesirable compounds, including nitrosamines or other mutagens. In certain embodiments, dosing and operating parameters—including component identity and ratios, temperature, fermentation duration, timing relative to meals, and sequencing relative to administration of fermented Component 5—are configured by a provider and/or pharmacist based on patient-specific factors and/or clinical data (e.g., adductomics) to support compatibility and tolerability in a given clinical use scenario.

In one embodiment, the approach may allow integration with existing therapeutic regimens without any significant clinical, pharmacodynamic interaction, or modification to the API. The approach may integrate with existing therapeutic regimens and APIs, avoiding any major chemical conversion of the medication prior to the point of dispensing or sale to the patient; the disclosure may facilitate a mutagen risk-mitigation approach of pharmacotherapy that is closer to, and focused on, the site of administration and/or absorption.

In some embodiments, the adjunct components are selected to avoid clinically meaningful interference with absorption, metabolism, distribution, or excretion of the therapeutic agent, and to avoid creating conditions that could increase formation or bioavailability of undesirable compounds, including nitrosamines or other mutagenic species. Thus, in certain embodiments, dosing and operating parameters (including component identity, component concentration ratios, temperature, fermentation duration, timing relative to meals, and adjunct scheduling relative to the fermented Component 5) are configured by a provider and/or pharmacist based on patient-specific factors and/or monitoring data to maintain therapeutic benefit and ADME while also mitigating mutagen exposure.

### 8.4.17 Advantages Over Prior Art

In certain embodiments, the disclosed protocols, systems, and methods provide one or more advantages relative to certain conventional approaches. For example, the disclosed embodiments may reduce exposure to nitrosamines and other mutagens without requiring major modification of the dispensed final drug product formulation or alteration of the shipment or storage. Improvement of the medicine may only occur at or near the site and time of administration and absorption. The approach may be applied across multiple therapeutic classes and dosage forms.

In addition, the disclosed embodiments provide flexible configuration of composition, dosing, and operating parameters that may be tailored to patient-specific and/or cohort-specific factors. In certain embodiments, the approach is compatible with existing regulatory and clinical workflows, wherein the PMM design and execution are configured or compounded based on applicable laws, selected combination of pharmaceutical parameters, fermentation conditions (including timing and temperature), dietary considerations, and/or medical history.

In some embodiments, the PMM and PMD systems are implemented in tandem with comprehensive medication management (CMM) and/or medication therapy management (MTM). In certain embodiments, such integration may improve operational efficiency and patient usability by bundling related services, which may reduce patient costs. In some embodiments, preparation and/or compounding of the fermented PMM mixture and configuration of the PMD are performed by a pharmacist, and the associated CMM and/or MTM services are also pharmacist-led patient care initiatives, thereby enabling a scalable, cost-effective, and efficient optimization of therapy and mutagen risk mitigation.

### 8.5.1 Scope and Support

The claims are supported by the detailed description, including multiple embodiments and prophetic examples set forth throughout this specification, which together provide sufficient written description and enablement for a person of ordinary skill in the art to make and use the disclosed compositions, systems, and methods without undue experimentation. The disclosed embodiments and examples illustrate representative formulations, process conditions, and adjunct combinations across a range of therapeutic agents and utilization cases, and are intended to be exemplary rather than limiting. Accordingly, variations, substitutions, modifications, and functional equivalents—such as changes in adjunct identity, concentration, ratios, sequencing of fermentation steps, and dosage-form configuration—may be implemented while achieving the described technical effects, and are contemplated as falling within the scope of the appended claims, to the extent consistent with this disclosure.

### 8.6 Examples

### Two Working Examples

### Working Example 1: Nitrite Test Strip Assessment Before and After PMD Fermentation for Oral Administration

The following working examples are provided mainly for the purpose of illustration and to describe representative embodiments that are expected to operate as described herein. The examples are not intended to be limiting and do not necessarily describe common embodiments of the present disclosure. Variations, modifications, substitutions, and combinations will be apparent to those of ordinary skill in the art and are intended to fall within the scope of the appended claims. Nitrites and nitrates are referenced as a representative nitrosation precursor and are not intended to limit the scope of mutagen risk mitigation addressed by the present disclosure. As noted by IARC, ingested nitrate or nitrite under conditions that result in endogenous nitrosation (e.g., under acidic gastric conditions) has been classified as probably carcinogenic to humans (Group 2A).

### Objective.

This example evaluated whether preparation of an exemplary PMM mixture using a PMD fermentation step reduced detectable nitrite in a PMM sample, using commercially available nitrite test strips.

### Materials.

(a) Bartovation nitrite test strips (colorimetric; UPC 649910801323); (b) drinking water (Poway, CA municipal tap water; FDA/EPA-compliant); (c) labetalol tablets, 100 mg ([Component 5]; e.g., NDC 50090-7458-0, exp. 07/2026); (d) Saccharomyces (e.g., S. boulardii and S. cerevisiae) [Component 3]; (e) cane sugar ([Component 4]; C&HQ; UPC 015800030126); and (f) bottled water (Kirkland; UPC 096619756803). A prototype fermenter of the PMD was used to maintain temperature during the fermentation period. Materials were prepared and used in accordance with the methods described herein.

### Procedure.

A baseline nitrite measurement was obtained by contacting a nitrite test strip with an unfermented control sample prepared by combining Poway, CA tap water with Components 5, and recording the indicated nitrite level by comparison to the manufacturer-supplied color chart. A test sample was then prepared by combining Components 3, 4, and 5 with Poway, CA tap water in the prototype PMD fermenter and allowing fermentation to proceed at approximately 28° C. for approximately 3 minutes. After completion of fermentation, a second nitrite measurement was obtained by contacting a new nitrite test strip with the fermented sample and recording the indicated level. This was done to assess whether exposure to the fermented sample altered strip performance; the same post-fermentation strip was subsequently contacted with municipal Poway, CA tap water as a confirmatory check. The procedure was repeated a third time.

### Controls.

The unfermented control sample was measured using the same test strip procedure. Poway, CA tap water was measured as a blank control, and bottled water was measured as an additional negative control to confirm expected strip response in the absence of detectable nitrite. Of note, “Not detected” indicated below the lowest reportable color block of the strip.

### Results.

In this working example, the unfermented control sample yielded a visible nitrite response on the test strip consistent with 1 ppm nitrite, utilizing the manufacturer's color scale and SOP, whereas the fermented sample produced no visible nitrite signal (i.e., nitrite was reduced to below the detectable limit of the strip under the fermented conditions). These observations demonstrated that, in at least one design of a working example, PMD-based fermentation of an exemplary PMM mixture reduced detectable nitrites relative to the unfermented control. In certain embodiments, lowering the detectable nitrite can be used as a representative indicator of diminished nitrosation potential at or near the site of administration, thereby supporting mitigation of mutagen exposure risk associated with pharmaceutical use.

| | Nitrite PPM | Nitrate PPM |
|---|---|---|
| Samples/control/blanks | (n = 3) | (n = 3) |
| Fermented PMM (labetalol) sample | <0.5 | <10 |
| Component 5 (API) aqueous sample | 1 | 100 |
| (unfermented standard labetalol drug) | | |
| Municipal tap water (Poway, CA) | 0.5 | 25 |
| (the blank) | | |
| Kirkland Bottled water (the Blank) | <0.5 | <10 |

### Working Example 2: Nitrite Test Strip Assessment Before and After PMD Fermentation for Topical Administration

### Objective.

This example evaluated whether preparation of an exemplary PMM mixture using a PMD fermentation step reduced detectable nitrite in a PMM sample, using commercially available nitrite test strips.

### Materials.

(a) Bartovation nitrite test strips (colorimetric; UPC 649910801323); (b) drinking water (Poway, CA municipal tap water; FDA/EPA-compliant); (c) Lidocaine cream 5% ([Component 5]; e.g., 373581000401); (d) Saccharomyces (e.g., S. boulardii and S. cerevisiae) [Component 3]; (e) cane sugar ([Component 4]; C&H®; UPC 015800030126); and (f) bottled water (Kirkland; UPC 096619756803). A prototype fermenter of the PMD was used to maintain temperature during the fermentation period. Materials were prepared and used in accordance with the methods described herein.

### Procedure.

A baseline nitrite measurement was obtained by contacting a nitrite test strip with an unfermented control sample prepared by combining Poway, CA tap water with Component 5, and recording the indicated nitrite level by comparison to the manufacturer-supplied color chart. A test sample was then prepared by combining Components 3, 4, and 5 with Poway, CA tap water in the prototype PMD fermenter and allowing fermentation to proceed at approximately 28° C. for approximately 3 minutes. After completion of fermentation, a second nitrite measurement was obtained by contacting a new nitrite test strip with the fermented sample and recording the indicated level. This was done to assess whether exposure to the fermented sample altered strip performance; the same post-fermentation strip was subsequently contacted with municipal Poway, CA tap water as a confirmatory check. The procedure was repeated a third time.

### Controls.

The unfermented control sample was measured using the same test strip method. Poway, CA tap water was measured as a blank control, and bottled water was measured as an additional negative control to confirm expected strip response in the absence of detectable nitrite. Of note, “Not detected” indicated below the lowest reportable color block of the strip.

### Results.

In this working example, the unfermented control sample yielded a visible nitrite response on the test strip consistent with 1 ppm nitrite, utilizing the manufacturer's color scale and SOP, whereas the fermented sample produced no visible nitrite signal (i.e., nitrite was reduced to below the detectable limit of the strip under the fermented conditions). These observations demonstrated that, in at least one design of a working example, PMD-based fermentation of an exemplary PMM mixture reduced detectable nitrites relative to the unfermented control. In certain embodiments, lowering the detectable nitrite can be used as a representative indicator of diminished nitrosation potential at or near the site of administration, thereby supporting mitigation of mutagen exposure risk associated with pharmaceutical use.

| | Nitrite PPM | Nitrate PPM |
|---|---|---|
| Samples/control/blanks | (n = 3) | (n = 3) |
| Fermented PMM (lidocaine cream) sample | <0.5 | <10 |
| Component 5 (API) aqueous sample | 5 | 250 |
| (unfermented standard lidocaine drug) | | |
| Municipal tap water (Poway, CA) | 0.5 | 25 |
| (the blank) | | |
| Kirkland Bottled water (the Blank) | <0.5 | <10 |

### Prophetic Examples

### 8.6.1 Iterative Threshold Value, Palatability Adjustment, and Clinical Action

### 8.6.1.a First Iteration

The following prophetic example(s) are provided for purposes of illustration and are not intended to be limiting. Unless otherwise specified, any parameter, component identity, component amount, ratio, processing step, comparator threshold, and endpoint may be selectable and adjustable based on the condition, the subject, the therapeutic agent (Component 5), and the selected adjunct components, while remaining within the scope of the claims.

In some embodiments, a care team (e.g., the subject and one or more providers and/or pharmacists) will configure an initial (“first-iteration”) PMM mixture and associated PMD operating parameters for use with a therapeutic agent (Component 5). In a representative first-iteration implementation, the care team will select a plant-derived Component 2 comprising pumpkin seed material and kale leaves (e.g., Brassicales plant material), and will prepare a baseline PMM mixture consisting of:
1. Component 1 (vitamin/supplement/antioxidant): ascorbic acid, fine powder, about 50 mg.
2. Component 2 (plant-derived material): pumpkin seed material and kale, wherein the kale will be blanched and will be combined with potable water and the pumpkin seed material to form a slurry, the slurry comprising a total plant mass of about 10 g to about 60 g (q.s.).
3. Component 3 (probiotic): Saccharomyces boulardii, about 1×10¹⁰ CFU.
4. Component 4 (nutrient/excipient): sucrose NF, about 4 g.
5. Component 5 (therapeutic agent): lisinopril, about 2.5 mg (e.g., NDC 00591-0405-01), or an equivalent lisinopril drug product.

Exemplary PMD operating parameters (illustrative only). In one prophetic implementation, a PMD fermenter will be filled with potable drinking water and will be maintained at a temperature of about 28° C. Components 1, 3, 4, and 5 will be combined in the fermenter, and fermentation will be allowed to proceed for about 1 minute. After the fermentation interval, the blended Component 2 slurry will be added to the fermenter and will be mixed. In certain embodiments, the order of addition will be varied (e.g., adding Component 2 prior to fermentation, during fermentation, or after fermentation).

Adductomics endpoint and decision rule (prophetic). In this prophetic example, a comparator threshold for an HSA adduct burden will be set at about 0.1 μmol adduct per mg HSA (maximum). Administration of the therapeutic agent in combination with the configured PMM mixture is expected to reduce one or more protein adduct metrics as assessed by an untargeted adductomics assay. By way of non-limiting example, during the first iteration the regimen will be configured such that an HSA adduct burden will be reduced toward a target value of approximately 0.5 μmol adduct per mg HSA, while a therapeutic benefit of Component 5 will be maintained (e.g., blood pressure goals will be set with the patient care team).

In one implementation, the care team will apply the following decision framework:
if the measured HSA adduct burden will not be at or below the comparator threshold for at least two consecutive measurements, then one or more parameters of the regimen will be adjusted (e.g., fermentation duration, temperature, pH, order of addition, adjunct identity, or adjunct amount); and
if the measured HSA adduct burden will be at or below the comparator threshold for at least two consecutive measurements, then the regimen will be maintained and/or one or more adjunct amounts will be decreased.

The foregoing endpoint and regimen parameters are illustrative and will be adjusted based on subject-specific response and/or monitoring data.

### 8.6.1.b Palatability Adjustment as an Iterative Input

In some embodiments, subject palatability parameters—including flavor, aroma, and texture—will be treated as measured or assessed inputs to an iterative PMM/PMD design workflow. Accordingly, a care team (e.g., the subject and one or more providers and/or pharmacists) will obtain palatability feedback according to a defined cadence (e.g., daily, weekly, or monthly) and, responsive to such feedback, will adjust one or more PMM formulation variables while maintaining the intended functional performance of the adjunct components.

In a representative prophetic example, the subject will report a preference for reduced flavor intensity and reduced particulate texture associated with the plant-derived Component 2. In response, the pharmacist will modify Component 2 by substituting, in whole or in part, the prior plant-derived material with a lower-texture plant-derived material comprising chia seed material, thereby reducing perceived texture and/or flavor while continuing to provide plant-derived constituents consistent with the disclosed embodiments and the intended PMM objectives.

### 8.6.1.c Iterative Protocol and Decision Framework: Second Iteration

In one embodiment, the decision framework for PMM adjustment will proceed as follows in this prophetic example. The patient care team will determine—based on adductomic and clinical monitoring—that an HSA adduct burden of approximately 0.5 μmol adduct per mg HSA will remain undesirably elevated relative to the comparator threshold, and that a blood pressure measurement (e.g., about 144/96 mm Hg) will warrant further improvement. In a second prophetic iteration, the patient care team will substitute Component 2 and will modify one or more fermentation parameters (e.g., temperature, time, and/or sugar concentration), while maintaining Component 1, modifying Component 3, and continuing administration of the therapeutic agent (Component 5).

In a representative second-iteration implementation, the PMM mixture will comprise:
1. Component 1: ascorbic acid, fine powder, about 50 mg.
2. Component 2 (plant-derived material): broccoli florets about 60 g, rinsed and trimmed to remove stalk material, and blended with chia seeds about 12 g and potable drinking water to form a slurry.
3. Component 3 (probiotic): Saccharomyces boulardii, about 4×10¹⁰ CFU.
4. Component 4 (nutrient/excipient): sucrose NF, about 6 g.
5. Component 5 (therapeutic agent): lisinopril, about 40 mg (e.g., NDC 00591-0409-05), or an equivalent lisinopril drug product.

Exemplary PMD operating parameters (illustrative only). In one prophetic implementation, the PMD fermenter will be filled with potable drinking water and will be maintained at a temperature of about 29° C.±2° C. Components 1, 3, 4, and 5 will be combined in the fermenter, and fermentation will be performed at approximately 29° C.±2° C. for about 4 minutes. Thereafter, the Component 2 slurry will be added and mixed, followed by oral administration.

Adductomics endpoint (prophetic). In this second iteration, the adjusted lisinopril dose, the substitution of broccoli and chia for Component 2, and the increased probiotic and fermentation settings will be expected to provide an improved reduction in one or more adductomics endpoints relative to administration of Component 5 without adjunct components, including reduction of an HSA adduct burden toward approximately 0.1 μmol adduct per mg HSA. This second-iteration regimen is also expected to improve blood pressure (e.g., toward about 135/80 mm Hg), depending on patient-specific factors, dietary patterns, and lifestyle variables. In such embodiments, baseline comparator data will include untargeted adductomics measurements obtained prior to initiating the regimen and/or during a baseline testing interval, and subsequent regimen adjustments will be made responsive to comparisons between the baseline comparator data and post-intervention adductomics results.

8.6.2 Iterative Percentage Adjustment Rule with Adductomic Comparator and Fermentation Time

In one prophetic embodiment, a subject will be administered a therapeutic agent (Component 5) in combination with one or more adjunct components. Biomarker data comprising untargeted adductomics endpoints will be obtained from a blood sample and will be processed to determine a total albumin adduct burden (e.g., total human serum albumin (HSA) adduct burden) using liquid chromatography-tandem mass spectrometry (LC-MS/MS). Comparator data will comprise baseline biomarker data for the subject that will be obtained prior to initiating administration of the therapeutic agent in combination with the adjunct component(s).

In this prophetic example, after a defined administration interval (e.g., about 90 days), the total albumin adduct burden will be compared to the baseline value to determine a percent change. If the total albumin adduct burden will have decreased by less than about 17% relative to baseline, a subsequent administration will be adjusted by increasing a fermentation duration for a subsequent fermented mixture (for example, increasing a fermentation duration from about 2 minutes to about 8 minutes), optionally while maintaining one or more additional fermentation parameters substantially constant. If the total albumin adduct burden will have decreased by at least about 19% relative to baseline, the fermentation duration will be maintained, and/or an amount of at least one adjunct component will be maintained (and optionally reduced in a maintenance phase).

In some embodiments, if the percent decrease will fall between about 17% and about 19%, a care team will optionally obtain an additional confirmatory measurement and/or apply one or more additional decision criteria (e.g., a clinical endpoint, an alternative biomarker, or palatability constraints) before maintaining or modifying fermentation settings.

### 8.6.3 Marketed Drug Product Compatibility: Clinical Knowledge of Drug-Drug Interaction

In one prophetic embodiment, a PMM mixture will be configured according to standard baseline parameters. A subject will be prescribed a marketed drug product comprising a therapeutic agent (Component 5), for example, mixed amphetamine salts extended-release 30 mg (e.g., NDC 0555-0974-02). A pharmacist and/or provider will identify potential vitamin-drug and/or food-drug interaction considerations for the subject's pharmacotherapy regimen. Based on professional judgment and subject-specific clinical parameters, a modified baseline PMM mixture will be selected to reduce interaction risk while maintaining compatibility with Component 5.

In a non-limiting prophetic example, Component 1 will be modified by replacing ascorbic acid (vitamin C) with an alternative antioxidant and/or stabilizing adjunct, for example, vitamin E and calcium carbonate (CaCO₃). Component 2 will be modified by replacing a kiwi and chia seed preparation with a soybean-based adjunct (e.g., soybeans, about 1 g) and a microbial-derived adjunct (e.g., spirulina, about 20 mg). The therapeutic agent will be combined with at least one adjunct component to form a mixture, and the mixture will be fermented under controlled fermentation parameters, including a defined fermentation duration, to produce a fermented mixture for administration.

In some embodiments, biomarker data comprising untargeted adductomics endpoints will be obtained from a blood sample and will be processed to determine a total albumin adduct burden (e.g., total HSA adduct burden) by LC-HRMS/MS. Comparator data will comprise baseline biomarker data for the subject obtained prior to initiating administration of the modified PMM regimen. After a defined administration interval (e.g., about 28 days) using the modified PMM mixture, the total albumin adduct burden will be expected to decrease relative to baseline and corroborated with FPM (for example, by about 49% adductomic burden and a trend of Hb P50 towards 26 in a representative prophetic outcome). In response, the fermentation duration and composition will be maintained for subsequent administrations, optionally subject to continued adductomic monitoring.

In such embodiments, the subject will be expected to maintain a desired therapeutic response to Component 5 (e.g., as assessed by a clinical score and/or symptom scale) while also exhibiting a reduction in one or more adductomics endpoints relative to baseline.

### 8.6.4 Algae-Derived or Microbial-Derived Materials

In one prophetic embodiment, a subject will be administered a therapeutic agent (Component 5) in combination with one or more adjunct components, wherein at least one adjunct component will comprise an algae-derived material and/or a microbial-derived material. In a representative prophetic implementation, the algae-derived material will comprise kombu, Irish sea moss, and/or sea grapes, and the microbial-derived material will comprise a microbial lysate, a cell wall fraction, and/or a probiotic comprising Bifidobacterium spp., Lactobacillus spp., and/or one or more microorganisms from the order Saccharomycesles. The therapeutic agent and at least one adjunct component will be combined to form a mixture, and the mixture will be fermented prior to administration.

Biomarker data comprising untargeted adductomics endpoints will be obtained from a blood sample and will be processed to determine a total albumin adduct burden by LC-HRMS/MS. Comparator data will comprise baseline biomarker data for the subject obtained prior to initiating administration. If, after a defined administration interval (e.g., about 28 days), the total albumin adduct burden will have decreased by less than about 17% relative to baseline, a subsequent administration will be adjusted by increasing a fermentation duration for the subsequent fermented mixture (e.g., increasing from about 2 minutes to about 8 minutes) and/or by increasing an amount of the algae-derived material and/or microbial-derived material within a predefined range. If the total albumin adduct burden will have decreased by at least about 19% relative to baseline, the fermentation duration will be maintained, and/or an amount of at least one adjunct component will be maintained or reduced in a maintenance phase, while adductomics endpoints will continue to be monitored and compared to baseline and/or to one or more target thresholds.

### 8.6.5 Example Formulation A—Probiotic—Vitamin Topical Cream

In one embodiment, a subject will be administered a therapeutic agent via a topical route in combination with one or more adjunct components, wherein the therapeutic agent will be fermented together with at least one adjunct component prior to administration. The following prophetic example is provided for illustration and is not limiting.

### Representative Prophetic Formulation (Cream).

Component 5 (therapeutic agent): diclofenac (e.g., diclofenac free acid or a pharmaceutically acceptable salt), present in the final topical cream at a therapeutically effective concentration (e.g., about 0.1% to about 5% w/w).
Component 1 (vitamin/antioxidant adjunct): vitamin E (tocopherol) and/or an ascorbic acid derivative, present in the final cream at about 0.01% to about 5% w/w.
Component 3 (probiotic adjunct): Saccharomyces boulardii and/or Bifidobacterium (e.g., B. longum, B. breve, and/or B. bifidum), provided as viable cells, present at an effective level (e.g., about 106 to about 10¹¹ CFU-equivalents per unit dose or per gram of mixture).
Optional Component 4 (fermentable substrate): sucrose, inulin, or another fermentable carbohydrate (e.g., about 0.1% to about 10% w/w).
Topical carrier: a pharmaceutically acceptable cream base (e.g., oil-in-water emulsion) optionally comprising emulsifiers, humectants, and stabilizers compatible with the adjunct component(s).

### Fermentation.

In one prophetic embodiment, diclofenac (Component 5) will be combined with at least one adjunct component (e.g., Component 1 and/or Component 3, optionally Component 4) to form a fermentable mixture, and the mixture will be fermented by controlling one or more fermentation parameters selected from duration, temperature, pH, light exposure, and order of addition. By way of non-limiting example, fermentation will be conducted at about 20° C. to about 37° C. for about 2 minutes to about 24 hours, optionally at a pH of about 4.0 to about 7.5, and with controlled light exposure. Following fermentation, the fermented mixture will be incorporated into the topical cream base to produce the diclofenac topical cream.

### Administration and Monitoring (Prophetic).

The fermented diclofenac topical cream will be administered according to a prescribed regimen. Biomarker data comprising one or more mutagen-related adductomic endpoints will be obtained according to a monitoring cadence and processed to determine a total adduct burden and/or a total albumin adduct burden by LC-MS/MS, using a biospecimen selected from blood, urine, saliva, and/or a local sample (e.g., a swab from a site of topical administration). Comparator data will comprise baseline biomarker data for the subject obtained prior to initiating administration and/or a threshold value.

If, after a defined interval (e.g., about 35 days), the Hb (hemoglobin) adductomic endpoint decreases by less than about 17% relative to baseline and/or remains above a threshold value, a subsequent administration will be adjusted by modifying at least one of: (i) an amount or form of the probiotic adjunct, (ii) an amount of the vitamin/antioxidant adjunct, (iii) a frequency or timing of topical application, and/or (iv) one or more fermentation parameters (e.g., increasing fermentation duration from about 2 minutes to about 8 minutes, adjusting fermentation temperature, adjusting pH, adjusting light exposure, and/or changing order of addition). If the Hb adductomic endpoint decreases by at least about 19% relative to baseline and/or reaches a target threshold, the fermentation parameters will be maintained as long as the Hb P50 O2 affinity is improved as determined by relative affinity error, deviation from 27 mmHg, and patient lifestyle.

### 8.6.6 Supplements as “Therapeutic Agent” (Component 5) With Adjunct Fermentation. (Phophectic)

### 8.6.6.1 Glucomannan Embodiment

In one embodiment, a subject will be managed under a patient-specific and/or cohort-specific regimen intended to support glycemic control and one or more cardiometabolic markers. The subject and a patient-care team (e.g., one or more providers and/or pharmacists) select a supplement to serve as the primary intended therapeutic agent for a defined therapeutic purpose. In a representative non-limiting prophetic example, Component 5 will comprise a glucomannan supplement, such as Glucomannan 1,800 mg capsules (UPC 810014679242). For purposes of this Section 8.6.6, the glucomannan supplement will be the therapeutic agent (Component 5) because it has a defined therapeutic purpose.

In some embodiments, palatability of the final preparation will be supported by one or more adjunct-carrier features providing an apple-cinnamon flavor profile. The patient-care team may evaluate safety, sourcing, compatibility, and contaminant considerations associated with cinnamon-containing and/or apple-derived materials and may select one or more suitable Component 2 materials accordingly.

In one prophetic embodiment, a PMM mixture will be prepared by combining and fermenting the glucomannan supplement (Component 5) with one or more adjunct components. In certain embodiments, the adjunct components comprise: (i) one or more probiotic adjuncts, including Saccharomyces boulardii and one or more Bifidobacterium species (e.g., B. longum, B. breve, and/or B. bifidum); (ii) one or more fermentation-support components, including sucrose and/or another suitable fermentable substrate; and, in some embodiments, (iii) one or more plant-derived adjuncts configured to support palatability and/or provide an apple-cinnamon profile. Fermentation will be performed ex vivo by dispersing Component 5 in water and controlling at least one fermentation parameter selected from duration, temperature, pH, light exposure, concentration, and order of adjunct component addition. By way of non-limiting example, fermentation may be conducted at about 28° C. to about 35° C. for about 5 minutes, optionally at a pH of about 4.0 to about 7.5, prior to administration of the fermented preparation.

### 8.6.6.1 (a) Monitoring and Decision Framework (Prophetic).

In one aspect of the disclosure, biomarker data comprising one or more untargeted and/or targeted adductomic endpoints will be obtained according to a monitoring cadence (e.g., weekly, monthly, or quarterly) and will be processed to determine a total adduct burden and/or a total albumin adduct burden (e.g., total HSA adduct burden) by LC-MS/MS and/or LC-HRMS/MS. Comparator data may comprise at least one of: (i) baseline biomarker data for the subject obtained prior to initiating the regimen; (ii) cohort biomarker data used to establish a population reference range; and/or (iii) one or more threshold values for one or more adductomic endpoints.

In some embodiments, additional clinical biomarkers relevant to the glucomannan regimen are also monitored, including fasting blood glucose, postprandial blood glucose, serum fructosamine, HbA1c, and/or one or more lipid biomarkers. In some embodiments, one or more functional protein metrics are additionally assessed as described elsewhere herein.

In some embodiments, one or more tolerability endpoints are additionally monitored, including abdominal discomfort, bloating, constipation, diarrhea, palatability-related nonadherence, and dose interruption. In certain embodiments, one or more safety endpoints, including suspected intestinal obstruction, are additionally monitored. Based on the tolerability and/or safety monitoring, the PMM will be fermented and administered using adjusted parameters, including fermentation duration, component identity, component amount and/or concentration, water volume, administration timing, and/or other PMM parameters, according to PMM instructions intended to support safe fermentation, safe administration, tolerability, and adherence. In some embodiments, tolerability endpoints and/or adverse drug events (ADEs) are monitored before and after the PMM intervention, and one or more incidence, severity, and/or risk measures are compared relative to baseline and/or comparator data to assess whether the PMM fermentation intervention is associated with improved tolerability and/or reduced ADE risk.

If, after a predefined interval (e.g., about 4 to 12 weeks), the total adduct burden has decreased by less than a predefined amount (e.g., less than about 12% relative to baseline) and/or remains above a threshold value, and/or one or more relevant clinical or tolerability endpoints fail to improve or worsen, subsequent administration may be adjusted by modifying at least one of: (i) an amount or ratio of S. boulardii to Bifidobacterium; (ii) fermentation duration, temperature, and/or pH; (iii) timing of administration of the fermented preparation relative to meals and/or other dietary nitrate/nitrite sources; (iv) selection or amount of one or more additional adjunct components; (v) palatability-supporting formulation variables; and/or (vi) administration instructions, including water volume and timing.

If the adductomic endpoints decrease by at least the predefined amount and/or reach a threshold goal, while one or more relevant clinical biomarkers move in a favorable direction and tolerability remains acceptable, the fermentation parameters and/or adjunct amounts may be maintained or reduced in a maintenance phase, while adductomic, FPM, and tolerability monitoring will be continuously performed longitudinally.

### 8.6.6.2 Glucomannan Supplement Capsule Implementation (Prophetic)

In a further prophetic embodiment, a PMM dosage form will be prepared using the following non-limiting components:
1. Component 1 (antioxidant adjunct): vitamin E, about 200 mg, selected to reduce nitrosation-related risk;
2. Component 2 (plant-derived adjunct): apple-skin puree, about 50 g; pulverized pumpkin seeds, about 17 g; cinnamon powder, q.s.; and/or one or more additional compatible plant-derived materials;
3. Component 3 (probiotic adjunct): S. boulardii and one or more Bifidobacterium species, totaling about 5×10\hat{ }8 CFU;
4. Component 4 (fermentation-support and/or chelating adjunct): calcium disodium EDTA (CaNa₂EDTA), about 50 mg, and sucrose NF, about 4 g; and
5. Component 5 (therapeutic agent): glucomannan, about 3,600 mg, for example Glucomannan capsules (UPC 810014679242).

In this embodiment, the capsule contents and/or separately packaged adjunct components are fermented either concurrently with Component 5, or in a partially separated fermentation workflow, by controlling one or more fermentation parameters as described herein. The resulting fermented preparation will be expected, in this prophetic embodiment, to decrease one or more markers associated with oxidative stress and/or adduct burden as assessed using biomarker monitoring including LC-MS/MS- and/or LC-HRMS/MS-based adductomics, while supporting palatability, adherence, and acceptable tolerability.

### 8.6.7 API-Only+Antioxidant/Buffer Fermentation (Cyanobacteria Microbes)

In one prophetic embodiment, a subject will be administered a therapeutic agent in combination with one or more adjunct components, wherein the one or more adjunct components will comprise an antioxidant adjunct and/or a buffering adjunct, and wherein the therapeutic agent will be subjected to a live-microbial fermentation step. In a representative prophetic implementation, the therapeutic agent will be combined with: (i) an antioxidant adjunct comprising ascorbic acid (vitamin C), about 100 mg, and tocopherol (vitamin E), about 100 mg; (ii) a buffering adjunct comprising sodium carbonate, about 5 mg; and (iii) one or more cyanobacterial and plant-derived adjuncts comprising sprouted chia seeds, about 16 g, chickpea beans, about 67 g, and live spirulina, about 90 g, to form a mixture. The mixture will be held under controlled fermentation, including a controlled pH of about 10.0 and light-exposure time, prior to administration. By way of non-limiting example, the buffering adjunct will be selected to provide a target pH range of about 8.5 to about 10, and the antioxidant adjunct will be selected to provide reactive oxygen species (ROS) scavenging and/or electrophile-trapping capacity during fermentation, preparation, and after administration. The light source will be especially vital in spirulina fermentation and will be timed at a duration of at least 16 minutes to about 8 hours.

Biomarker data comprising untargeted adductomics endpoints will be obtained from a blood sample and will be processed to determine a total albumin adduct burden (e.g., total HSA adduct burden) by LC-HRMS/MS. Comparator data will comprise baseline biomarker data for the subject obtained prior to initiating administration. If, after a defined interval (e.g., about 28 days), the total albumin adduct burden will be determined to have decreased by less than about 17% relative to baseline, a subsequent administration will be adjusted by modifying one or more fermentation and/or preparation parameters, including, without limitation: increasing a hold time for the mixture (e.g., from about 20 minutes to about 36 hours); adjusting an amount of the buffering adjunct to move the mixture pH toward the target range; increasing an amount of the antioxidant adjunct within a predefined range; modifying light-exposure duration; and/or changing the order of addition such that the antioxidant adjunct and the buffering adjunct will be combined prior to introducing the therapeutic agent. If the total albumin adduct burden will be determined to have decreased by at least about 19% relative to baseline, and Hb P50 will be determined to have moved toward a target value of about 24-27 mm Hg, then the fermentation and/or preparation parameters will be maintained, and/or an amount of at least one adjunct component will be maintained or reduced, while adductomics endpoints will continue to be monitored.

### 8.6.8 Dosing Based on Body Weight

In some embodiments, when preparing or fermenting the PMM formulation, a baseline dose of one or more adjunct components will be adjusted, at least in part, based on patient body weight. By way of example:
Patients weighing <50 kg: 150 mg of Vitamin C (antioxidant, Component 1) and 5×10\hat{ }8 CFU of probiotics (Component 3).
Patients weighing 50-75 kg: 200 mg of Vitamin C (antioxidant, Component 1) and 1×10\hat{ }9 CFU of probiotics (Component 3).
Patients weighing >75 kg: 350 mg of Vitamin C (antioxidant, Component 1) and 3×10\hat{ }9 CFU of probiotics (Component 3).

In further embodiments, the baseline adjunct composition and/or dose selection will be additionally determined based on one or more of: family history, pharmacogenetic profile, prior side effects or adverse events, pharmacodynamic response, medical history, and patient preferences.

### 8.6.9 Timing Relative to Meals

In some prophetic embodiments, the timing of PMM administration relative to food intake will be configured to modulate adjunct performance and to attenuate nitrosation reactions associated with meal-derived precursors. By way of example:
Pre-meal administration: The PMM will be administered about 30-60 minutes prior to a meal, such as a high-protein meal, to mitigate nitrosation reactions that may be enhanced by dietary amines.
With-meal administration: The PMM will be administered concurrently with a meal to promote mixing with gastric contents and/or to provide sustained adjunct activity during digestion.
Targeted with-meal administration for higher-risk meals: The PMM will be administered concurrently with meals that include one or more of processed meats, smoked meats, salted or cured meats, grilled meats, preserved or pickled foods, breads, and/or alcohol to attenuate formation of nitrosation byproducts associated with such dietary exposures.
With-meal administration with plant-derived foods: In certain embodiments, the PMM will be administered with meals containing cruciferous vegetables and/or other plant-derived materials, which may influence gastric pH and/or redox conditions and thereby potentially enhance adjunct performance.

### 8.6.10.1 Pediatric Embodiments (Palatability and Age-Appropriate Dosing)

In some embodiments, PMM formulations and dosing regimens are adapted for pediatric and geriatric populations to improve tolerability, adherence, and clinical performance, including reducing adverse event risk.

In certain embodiments, the flavor-masking approach is selected based on a dominant taste profile of the formulation:
Bitter: chocolate, licorice, or coffee flavorants.
Salty: butterscotch, cinnamon, or peanut-butter flavorants.
Sour: citrus flavorants.

The PMM may be provided as a fermented syrup or suspension comprising flavor-masking excipients, Component 3, Component 4, and Component 5.

By way of example, probiotic dosing (Component 3) may be tiered by age:
Age 3 months to 3 years: 5×10\hat{ }7 CFU of Saccharomyces boulardii (probiotics Component 3).
Age 3 years to 9 years: 1×10\hat{ }8 CFU of Saccharomyces boulardii (probiotics Component 3).
Age ≥9 years: 2×10\hat{ }8 CFU of Saccharomyces boulardii (probiotics Component 3).

In some embodiments, pediatric dosing will be additionally adjusted based on body weight and/or clinical status.

### 8.6.10.2 Geriatric Embodiments (Polypharmacy, Adherence, and Renal Function)

In some embodiments, PMD administration for geriatric patients will be coupled with a monitoring and support workflow to address polypharmacy risk, adherence, adverse events, and logistics associated with preparing and delivering the PMM. In certain embodiments, unit-dose packaging, pre-measured sachets, and/or daily delivery will be provided to improve fermentation, adherence, and reduce preparation errors.

In some embodiments, antioxidant dosing (Component 1) will be adjusted to maintain efficacy when used with fermentation processes and concomitant medications, including adjusting the dose upward within a tolerated range when extensive fermentation will be anticipated.

In certain embodiments, patients receiving medications associated with increased geriatric risk (e.g., medications identified by the Beers Criteria) are assigned (i) a longer fermentation duration and/or (ii) higher probiotic concentrations, to increase detoxification and/or impurity attenuation performance while decreasing risk of adverse events.

By way of example, probiotic dosing (Component 3) will be adjusted based on renal function in patients aged 55 years and older:
eGFR 90-60 mL/min/1.73 m²: 5×10\hat{ }10 CFU.
eGFR 60-30 mL/min/1.73 m²: 8×10\hat{ }12 CFU.
eGFR 30-10 mL/min/1.73 m²: 6×10\hat{ }14 CFU.

### 8.6.11 Comorbidity-Specific Adjustments and Contraindications

In some embodiments, the provider will configure the PMM/PMD protocol based on patient comorbidities and physiologic reserve.
Renal impairment: The provider will have the option to reduce the dose and/or frequency of chelating agents to mitigate risk of systemic accumulation and electrolyte disturbances, and may increase monitoring of renal function and relevant laboratory parameters during administration.
Hepatic impairment: The provider will have the option to adjust antioxidant selection and relative ratios to reduce hepatic oxidative burden and avoid hepatically burdensome excipients or adjuncts, optionally titrating dosing based on liver function tests and clinical tolerance.
Acute severe illness/critical care: Acute severe illness, ICU admission, hemodynamic instability, active sepsis, and/or the presence of indwelling vascular access (e.g., a central venous catheter) may constitute relative or absolute contraindications to initiating or continuing PMM/PMD execution due to heightened infection risk, altered pharmacokinetics, and reduced physiologic reserve. Because the disclosed protocol requires ex vivo fermentation of the therapeutic agent prior to administration, the provider may defer initiation or continuation of the PMM/PMD protocol until clinical stabilization and reassessment of suitability.

### 8.7.0 Further Embodiments

### 8.7.1 Controlled-Release Adjunct Components

In some embodiments, one or more adjunct components (e.g., antioxidants, chelating agents, plant-derived materials, probiotics, prebiotics, and/or combinations thereof) are formulated as controlled-release multiparticulates (e.g., granules, pellets, beads, or microcapsules) in which a probiotic and/or other microbial payload is embedded within, adsorbed onto, entrapped within, and/or coated by a polymer matrix. The matrix may comprise one or more food-grade and/or pharmaceutically acceptable polymers, including, by way of non-limiting example, alginates, pectins, chitosan-containing coatings, cellulose derivatives (e.g., hypromellose), and/or methacrylate copolymers. In certain embodiments, the multiparticulate format protects the adjunct payload from gastric acidity, bile exposure, moisture, and oxygen and regulates release by diffusion, swelling, erosion, pH-responsive release, and/or enzymatic disassembly. In exemplary embodiments, the multiparticulates provide a sustained-release profile over about 2 to about 8 hours (e.g., about 4 to about 6 hours) to maintain luminal exposure and preserve activity across upper-GI transit, consistent with microencapsulation and coating strategies for probiotic stability and controlled release. In certain embodiments, the therapeutic agent has already undergone the required ex vivo fermentation prior to administration, and the controlled-release formulation is configured to preserve, transport, and/or release one or more adjunct components after administration. Thus, the controlled-release dosage form may extend adjunct availability and/or adjunct-associated activity in the gastrointestinal tract following the initial ex vivo fermentation step, without requiring that Component 5 itself undergo in vivo fermentation.

8.7.2 pH-Responsive Capsules

In some embodiments, the formulation will be packaged in a pH-responsive (e.g., enteric) capsule or coating configured to remain substantially intact under gastric conditions (e.g., pH ≤about 5) and to dissolve and/or become permeable upon exposure to the higher pH associated with the small intestine (e.g., pH ≥about 5.5-7). This configuration will bias probiotic release to the small intestine and will reduce acid-mediated viability loss in the stomach. Non-limiting examples of enteric technologies will include methacrylic acid copolymer coatings and hypromellose-based acid-resistant capsules and/or coatings, including multilayer and/or triggerable microcapsule architectures that will disassemble in response to intestinal conditions (e.g., pH and/or enzymatic triggers).

8.7.3 Combination with Nutraceuticals

In some embodiments, one or more adjunct components will be combined with one or more nutraceuticals that support microbiome function, including prebiotic fibers such as inulin and fructooligosaccharides (FOS), thereby providing a synbiotic configuration. The prebiotic will be co-formulated (including, in some embodiments, co-encapsulation) with the probiotic to improve survival during processing and gastrointestinal transit and to promote downstream enrichment and/or engraftment of beneficial taxa after release. In such embodiments, the synbiotic formulation will be selected and configured to influence gut microbiota composition and one or more inflammatory and/or metabolic markers, with outcomes expected to depend on strain selection, dose, and host context. Accordingly, in some embodiments, the type and amount of the prebiotic (e.g., gram-range daily equivalents) will be tailored to the selected probiotic and to the intended patient-specific clinical use.

### 8.7.4 Modular Dispensing Devices

In some embodiments, a modular dispensing device will be provided that will house a plurality of interchangeable cartridges (or pods) containing different adjunct components (e.g., a Component 1 antioxidant, a Component 2 plant-derived material in fresh and/or pulverized form, a Component 3 probiotic, a Component 4 chelating agent, and/or one or more optional excipients). The device will be configured such that a patient and/or provider will select one or more cartridges based on a medication schedule, a current PMM/PMD iteration, and/or an updated care plan.

In certain embodiments, the device will comprise a user interface and/or controller (e.g., a QR-code reader, near-field communication interface, touch display, and/or physical controls) configured to provide electronic prompts and/or printed output (e.g., time-based reminders, stepwise workflow instructions, and alerts for contraindications or required deferral) and to perform dose tracking and audit logging, including recording a cartridge identifier, lot number, dispensing timestamp, and dispensed quantity. In some embodiments, the device will further support smartphone integration (e.g., via a companion application) to display prompts, capture confirmations, transmit adherence logs, and enable remote configuration by a provider and/or pharmacy.

In some embodiments, the controller will execute rule-based logic and/or an algorithmic decision framework to recommend cartridge selection and sequencing (e.g., “Component 1, 3, 4, 5 at time T1; Component 2 at time T2”), optionally based on medication administration times, patient preference, prior iteration results, and/or mixture viscosity and concentrations. In further embodiments, the device will synchronize, subject to permissions, with one or more external systems including a mobile application, a pharmacy information system, an MTM/CMM module, and/or an electronic health record (EHR) to support documentation, protocol updates, and longitudinal adherence monitoring.

### 8.8 Evaluation and Iterative Verification of Mutagen Mitigation

### 8.8.0 Evaluation and Iterative Verification

In some embodiments, the protocol will be evaluated using any suitable in vitro and/or in vivo measurement to verify, estimate, or otherwise infer a risk reduction in mutagenic burden and to guide subsequent iterations. The analytical platform is not limiting and may include validated LC-MS/MS or HRMS methods, immunoassays, enzymatic assays, and/or commercially available clinical laboratory tests.

### 8.8.1 Biomarker Monitoring and Decision Thresholds

In some embodiments, clinical evaluation includes measuring one or more biomarkers associated with nitrosation stress, oxidative DNA damage, and/or nitroso compound exposure (e.g., urinary 8-hydroxy-2′-deoxyguanosine (8-OHdG), urinary or plasma N-nitroso compounds, BO-Alb, Me-Cys34, or other validated markers). A reduction relative to baseline and/or a threshold comparator supports efficacy and may trigger maintenance dosing, whereas failure to improve may trigger a subsequent protocol iteration (e.g., adjusting Component 1 dose, substituting Component 2 botanical class, increasing Component 3 CFU, modifying fermentation time/temperature, or altering administration timing relative to the therapeutic agent).

### 8.8.2 Clinical Evaluation and Real-World Assessment

In some embodiments, clinical evaluation will be performed to assess standard therapy alone versus standard therapy supplemented with an adjunct composition and/or execution of the PMM. Non-limiting measures may include: (i) the occurrence, frequency, and/or severity of functional protein deviations and metrics, (ii) adverse events and tolerability; (iii) change from baseline in one or more biomarkers associated with adductomic stress and/or genotoxicity; (iv) chart review, patient-reported outcomes, laboratory values, and (v) confirmation that the therapeutic response to the primary drug is maintained (e.g., absence of non-efficacy), and patient preference in use, thereby supporting compatibility of the adjunct composition(s) with the patient's medication regimen.

### 8.8.3 Adductomic Reduction Supported by Functional Protein Metrics

In some embodiments, clinical evaluation and iterative redesign of the PMM and/or PMD are validated, corroborated, and/or guided by measurements demonstrating that one or more functional protein metrics have moved toward a predefined target value and/or shifted favorably relative to a subject-specific baseline, a matched-cohort comparator, and/or a population reference range. Non-limiting examples of functional protein metrics include: (i) hemoglobin oxygen affinity, including P50; (ii) coagulation-associated measurements, including INR and/or whole-blood clotting time (WBCT); (iii) a ratio, slope, trend, and/or other relationship between change in ferritin and/or transferrin and change in hemoglobin over a defined interval, including Δferritin/Δhemoglobin; (iv) error, bias, deviation statistics, normalized ranges, and/or discordance between HbA1c-derived estimates of average glucose and a longitudinal average of directly measured blood glucose values; and/or (v) other calculated and/or directly measured biochemical indices reflecting protein function, protein-associated physiologic performance, and/or protein modification. In certain embodiments, improvement in one or more such functional protein metrics, alone or in combination with one or more adductomic endpoints, supports a determination that the PMM composition, PMD operating parameters, administration timing, fermentation conditions, and/or monitoring intervals are appropriately configured or should be further adjusted.

### 8.9 Embodiments with Industrial Applicability and Lamotrigine

### 8.9.1 Manufacturing and Packaging

In some embodiments, adjunct compositions and/or kit components will be manufactured using conventional pharmaceutical and/or nutraceutical processes, including non-limiting examples of blending, milling, granulation, drying, tableting, encapsulation, sachet filling, and/or unit-dose packaging. In embodiments comprising probiotics, manufacturing and packaging may be configured to preserve viability by controlling moisture and oxygen exposure and by employing protective formats such as high-barrier blisters, foil laminates, desiccant-containing containers, and/or temperature-controlled handling and distribution. In kit-based embodiments and PMD utilization, individual components may be packaged in separate compartments or cartridges and assembled using existing filling and packaging lines with minimal modification.

### 8.9.2 Regulatory Pathways (Non-Limiting)

In some embodiments, the adjunct composition and/or an associated PMM kit, system, or device will be subject to different regulatory classifications depending on jurisdiction and on factors including, without limitation: intended use, labeling and marketing claims, route of administration, dosage form, product configuration (e.g., standalone adjunct, co-packaged product, co-administered product, or device-associated delivery), and whether the product is supplied as a finished article or prepared on demand.

In various non-limiting embodiments, the adjunct composition and/or PMM kit, system, or device will be classified and/or regulated as one or more of the following: a compounded preparation, a compounded medication, a dietary supplement, a food or medical food, an over-the-counter product, a prescription drug product, a medical device (or device component), a drug-device combination product, and/or a regulated accessory to a drug product.

### 8.9.2.1. Regulated Compounded Embodiments

In some embodiments, the fermented PMM will be provided as a compounded medication prepared by, or under the supervision of, a licensed pharmacy or healthcare facility in accordance with applicable local requirements. In certain embodiments, the compounded PMM will be prepared and fermented based on a patient-specific or cohort-specific prescription and administered solely by the patient. In certain embodiments, the compounded PMM will be fermented for office/clinical use where permitted.

### 8.9.2.2 Regulated Kit, Co-Pack, and Device-Associated Embodiments

In some embodiments, the PMM and/or PMD will be provided as a kit comprising (i) the adjunct composition in a pre-measured unit dose, multi-dose container, or blister configuration and (ii) instructions for use describing co-administration with a primary fermented pharmaceutical agent. In some embodiments, the kit further comprises one or more of: fermentation instructions, measuring devices, light sources, temperature readers, dosing calendars, patient information inserts, child-resistant packaging, desiccants/oxygen scavengers, or tamper-evident components.

In some embodiments, the PMM will be co-packaged with a primary pharmaceutical agent (e.g., in a co-pack, combination pack, or synchronized blister pack) while the adjuncts will remain distinct from the primary pharmaceutical agent. In some embodiments, the PMM includes or interfaces with a device component (e.g., a dispenser, applicator, metering component, cartridge, light sources, temperature readers, syringe, pump, smart cap, or compliance monitor) configured to facilitate fermentation, dosing, adherence, timing, and/or traceability.

### 8.9.3 Exemplary Adductomic, Functional Protein Metric, and Tolerability-Guided Iterative Adjustment Rule for Lamotrigine (Prophetic)

In certain embodiments, Component 5 will be fermented ex vivo, outside the body, and prior to administration; one or more fermented adjunct components are selected for mutagen mitigation, compatibility, tolerability, and/or adherence; and the regimen will be maintained or adjusted based on adductomic endpoints and, in some embodiments, one or more functional protein metrics, tolerability endpoints, adverse drug events (ADEs), and/or other clinical data.

In one prophetic embodiment involving lamotrigine, the patient-care team configures PMM and/or PMD in view of known clinical rash-risk considerations, including titration schedule, concomitant valproate exposure, and prior rash history with other antiepileptic drugs. Without being bound by theory, selected adjuncts may reduce reactive-metabolite burden in a subset of subjects and may be evaluated for association with improved tolerability. Comparator data may comprise a standard titration regimen without PMM/PMD. Non-limiting tolerability endpoints may include dose interruption, discontinuation, time to first cutaneous adverse event, and/or incidence or severity of non-serious cutaneous reactions, while standard clinical precautions for lamotrigine rash remain applicable.

In one prophetic embodiment, a fermented PMM comprises lamotrigine as the therapeutic agent (Component 5) administered according to a patient-specific regimen together with one or more adjunct components selected for compatibility, adherence, palatability, and potential attenuation of nitrosating species and/or other reactive intermediates associated with Component 5.

Without being bound by theory, one or more adjunct components may function through nitrosation inhibition and sequestration, electrophile scavenging, mutagen sequestration and/or inactivation, reduction of adduct burden through provision of a bio-nucleophilic sink, and/or microbiome modulation. In certain embodiments, nitrosamine formation can be promoted under acidic conditions when nitrosating species derived from nitrite contact amine-containing compounds; accordingly, consumption, neutralization, and/or sequestration of nitrite and/or nitrosating species may reduce mutagenic risk. In certain embodiments, standard clinical precautions applicable to lamotrigine-associated rash remain in effect, including recommended titration practices, caution with concomitant valproate, and prompt clinical evaluation of concerning cutaneous findings.

### 8.9.3 (a) Representative Prophetic Composition (Non-Limiting):

(i) Component 1: one or more antioxidants and/or polyphenols, including, by way of non-limiting example, ascorbic acid at about 50 mg to about 60 mg;
(ii) Component 2: one or more plant- and/or algae-derived adjuncts, including, by way of non-limiting example, chia seed material, flaxseed material, kiwi material, and/or sea-grape material, q.s.;
(iii) Component 3: one or more probiotic adjuncts provided as viable cells, including, by way of non-limiting example, Saccharomyces boulardii and/or Bifidobacterium, present at about 10\hat{ }8 to about 10\hat{ }10 CFU-equivalents per dose;
(iv) Component 4: one or more prebiotic and/or fermentation-support components, including, by way of non-limiting example, inulin at about 2 g, sucrose at about 1 g to about 2 g, and/or a buffering adjunct such as disodium phosphate; and
(v) Component 5: lamotrigine, for example an oral lamotrigine dosage form such as a 200 mg tablet, in a form suitable for ex vivo fermentation prior to administration.

In certain embodiments, Component 5 is fermented ex vivo together with at least one adjunct component to form a fermented PMM composition. In other embodiments, Component 5 is fermented separately from one or more adjunct components and is then combined with the separately fermented adjunct composition or administered in coordinated temporal relation thereto. Fermentation may be performed by controlling one or more parameters selected from duration, temperature, pH, light exposure, concentration, substrate availability, and order of addition, as otherwise described herein.

### 8.9.3 (b) Monitoring and Iterative Adjustment (Prophetic):

Biomarker data comprising one or more mutagen-related adductomic endpoints are obtained according to a predefined monitoring cadence and processed to determine total adduct burden and/or total albumin adduct burden by LC-MS/MS and/or LC-HRMS/MS using a biospecimen selected from blood, urine, saliva, and/or another clinically appropriate sample. Comparator data comprises subject-specific baseline biomarker data, matched-cohort data, and/or one or more threshold values. In some embodiments, one or more functional protein metrics are additionally assessed according to a predefined assay, comparator, and decision rule, including, by way of non-limiting example, hemoglobin oxygen affinity (P50), INR, WBCT, a predefined ferritin/transferrin/hemoglobin relationship, and/or HbA1c discordance.

In certain embodiments, one or more potential contributors to tolerability findings and/or adverse drug events (ADEs) are investigated, including one or more lamotrigine-metabolite measures such as lamotrigine-N-oxide, which may be evaluated as potential mechanistic root causes, without requiring any particular metabolite to be established as causal for rash. Without being bound by theory, such investigations may provide evidence regarding whether lamotrigine metabolism, including lamotrigine-N-oxide formation, is associated with increased rash occurrence in a subset of subjects, and whether ex vivo fermentation of lamotrigine in the PMM is associated with potential improvements in prevention, metabolite-related measures, adductomic burden, and/or tolerability endpoints. Any such association may be treated as value added for the literature's mechanistic data, but does not replace standard lamotrigine titration, patient-centered care, and rash-monitoring precautions.

If, after a defined interval (e.g., about 28 days), total albumin adduct burden is determined to have decreased by less than about 17% relative to baseline, and/or one or more functional protein metrics fail to move toward a predefined target value, and/or one or more tolerability endpoints worsen or fail to remain acceptable, subsequent administration may be adjusted by modifying at least one of: (i) an amount, identity, or form of the probiotic adjunct; (ii) an amount or identity of the prebiotic adjunct and/or antioxidant, plant-derived, and/or algae-derived adjunct; (iii) timing of administration of the fermented PMM relative to meals and/or Component 5; (iv) one or more formulation parameters, including ingredient composition, concentrations, polyphenol load, and/or palatability-support variables; and/or (v) one or more preparation parameters, including hold time, fermentation duration, temperature, pH, light exposure, buffering adjunct amount, antioxidant adjunct amount, and/or order of addition.

In some embodiments, if total albumin adduct burden is determined to have decreased by at least about 19% relative to baseline, and hemoglobin P50 is determined to have moved toward a target range of about 24 mm Hg to about 27 mm Hg, while tolerability remains acceptable and no clinically concerning cutaneous signal is identified, then the formulation and/or fermentation parameters may be maintained while adductomic endpoints and, where applicable, one or more functional protein metrics continue to be monitored. In certain embodiments, maintenance may additionally be supported by improvement in one or more additional functional protein metrics, including INR, WBCT, a predefined ferritin/transferrin/hemoglobin relationship, and/or reduced discordance between HbA1c-derived estimated average glucose and longitudinally averaged directly measured blood glucose values.

In embodiments in which a correlation is observed between improved adductomic endpoints and improved tolerability, the PMM regimen may be maintained and further monitored. However, no particular mechanistic correlation is required, and standard lamotrigine safety precautions remain applicable throughout the regimen. Illustrative non-limiting outcomes may include reduction in one or more measured adductomic endpoints relative to baseline and, in some embodiments, improved tolerability relative to baseline and/or comparator data. Such outcomes are illustrative, prophetic, and non-limiting and do not limit the claimed subject matter.

### 8.10 Exemplary Embodiments in Which Supplements Will Serve as Therapeutic Agents

In some embodiments, one or more supplements and dietary regimens will be associated with formation and/or presence of electrophilic species and/or potentially mutagenic compounds, including without limitation nitrosamines, acrylamide, and/or acetaldehyde, including under storage conditions, preparation and/or cooking conditions, and/or shipping conditions. In some embodiments, the systems and methods described herein will be used to reduce one or more mutagen-related adductomic endpoints associated with such exposures and will be coupled with the improvement of one or more functional protein metrics.

### Supplements PMM Embodiments and Adjunct Integration Formats

In some embodiments, a PMM adjunct composition will be incorporated into, co-packaged with, and/or co-administered with a supplement to provide a convenient, patient-acceptable delivery format that will support fermentation, adherence, and/or personalization.

Supplements dietary PMM embodiments will include the following:
1. Beverage embodiments: the adjunct composition will be provided in a ready-to-drink beverage, a concentrated shot, a powder packet for reconstitution, or an ORS/electrolyte solution.
2. Bar embodiments: the adjunct composition will be incorporated into a nutrition bar, protein bar, fiber bar, or GI-support bar.
3. Gel embodiments: the adjunct composition will be incorporated into a glucose gel, carbohydrate gel, or GI-support gel.
4. Enteral embodiments: the adjunct composition will be incorporated into an enteral formula, or will be provided as a separate whole-food or processed-food add-in compatible with enteral administration protocols.
5. Fiber/prebiotic embodiments: the adjunct composition will be co-formulated with dietary fibers and/or prebiotics.
6. Probiotic embodiments: the PMM composition will consist of one or more probiotics and/or synbiotic systems, including fermented or fermentation-compatible matrices, or shelf-stable probiotic formats.
7. Meal replacement embodiments: the adjunct composition will be included in a meal replacement designed for patient-centered diets, low-FODMAP dietary protocols, renal or hepatic support diets, or other nutrition regimens.
8. Whole-food embodiments or processed-food embodiments: the adjunct composition will be provided as a seasoning, coating, topping, spread, or sachet intended for co-ingestion with one or more food items.
9. Co-administration embodiments: the food product, adjuncts, and PMM compositions will be fermented prior to consumption.

In some embodiments, the supplement PMM adjunct composition will comprise one or more of: probiotics, prebiotics, antioxidants, polyphenols, nucleophilic scavengers, nitrosation inhibitors, binders and/or adsorbents, vitamins, supplements, pH-modulating agents, and/or combinations thereof, as described elsewhere herein.

### 8.10.1 Administration and Monitoring Workflow for Supplement PMM

In some embodiments, a supplement PMM (e.g., natural medicines) will be administered to a subject according to a prescribed or recommended regimen, including co-administration with one or more adjunct components and administration within a defined fermentation timing window relative to a fermented Component 5.

In some embodiments, biomarker data comprising one or more mutagen-related adductomic endpoints will be obtained according to a monitoring cadence and processed to determine a total adduct burden and/or a total albumin adduct burden using LC-HRMS/MS. In various embodiments, one or more functional protein metrics will additionally be obtained, including, without limitation: hemoglobin oxygen affinity (P50); INR; whole-blood clotting time (WBCT); a ratio, slope, trend, and/or other relationship between change in ferritin and/or transferrin and change in hemoglobin over a defined interval; error, bias, deviation, and/or discordance between HbA1c-derived estimates of average glucose and longitudinal averages of directly measured blood glucose values; and/or other calculated or measured biochemical indices of protein function. In various embodiments, the biospecimen will be selected from blood, urine, saliva, stool, buccal cells, and/or other clinically acceptable samples. Comparator data will comprise: (i) baseline biomarker data for the subject or cohort obtained prior to initiating the dietary PMM intervention; and/or (ii) a threshold value.

### 8.10.2 Exemplary Dietary Supplement Integration (Fermented Nutrition Bar and Co-Administration Embodiments)

A fermented supplement nutrition-bar embodiment comprising a probiotic may be formulated as part of a patient-specific dietary regimen to deliver the probiotic therapeutic agent together with one or more fermented PMM adjunct components in a convenient and palatable format that supports adherence. In all embodiments of this Section 8.10, fermentation is performed ex vivo prior to administration.

8.10.3 Exemplary Supplements Oral PMM with Ketogenic-Compatible (Non-Limiting)

In one non-limiting embodiment, a fermented PMM comprises an oral supplement therapeutic agent administered together with a custom nutritional drink that serves as an adjunct carrier and/or co-administered nutritional matrix. Component 5 will be the oral supplement therapeutic agent, and the drink provides one or more adjunct components and optional carrier material selected to support palatability, adherence, and compatibility with a patient-specific dietary regimen. In certain embodiments, the dietary regimen will be ketogenic or ketogenic-compatible. In all embodiments of this Section 8.10, the therapeutic agent (Component 5) will be fermented ex vivo prior to administration.

An oral PMM composition with a keto-supplement as Component 5 (non-limiting):
(i) Component 3 adjunct component(s): one or more probiotics provided as viable cells, including, by way of non-limiting example, S. boulardii and Bifidobacterium, optionally totaling about 7×10\hat{ }12 CFU;
(ii) Component 4: one or more prebiotic and/or fermentation-support components, including, by way of non-limiting example, inulin at about 2 g. In certain embodiments, Component 4 further comprises a fermentable carbohydrate and/or excipient, including, by way of non-limiting example, sucrose at about 2 g. In certain ketogenic-compatible embodiments, the fermentable carbohydrate component may be reduced, omitted, or replaced with another patient-specific fermentation-support component;
(iii) Component 1: one or more antioxidants and/or polyphenols, including, by way of non-limiting example, ascorbic acid at about 60 mg;
(iv) Component 2: one or more nutritional-matrix and/or plant-derived components configured for a drink composition, including, by way of non-limiting example, pea protein and pumpkin seed material, maple syrup and/or almond butter q.s.
(v) Component 5: a MCT supplement serving as the oral therapeutic agent comprising, by way of non-limiting example, MCT Oil Softgels 1000 mg, with UPC 857077008688, and will be supplied in a suitable form for ex vivo fermentation prior to administration.

### 8.10.3.b Administration:

In certain embodiments, Components 1-5 are combined and fermented concurrently ex vivo to form a final drink composition.

In alternative embodiments, Components 1~4 are first combined and fermented ex vivo to form a pre-fermented adjunct drink composition, and Component 5 will be separately fermented ex vivo prior to administration.

In such alternative embodiments, the separately fermented Component 5 will be administered concurrently with the pre-fermented adjunct drink composition or within about 2 hours after completion of fermentation of Component 5.

In further embodiments, the separately fermented Component 5 will be admixed with the pre-fermented adjunct drink composition immediately before administration or after completion of the respective fermentation steps.

In certain embodiments, the final fermented PMM drink composition will be administered promptly after completion of fermentation. In other embodiments, the final fermented PMM drink composition will be stored under refrigeration in an appropriate food-safe and/or pharmaceutically suitable container and assigned a beyond-use date of about 14 days, subject to applicable stability, microbial, and handling controls.

### 8.10.3.c Monitoring and Comparator:

Biomarker data comprising one or more mutagen-related adductomic endpoints will be obtained according to a predefined monitoring cadence and processed to determine total adduct burden and/or total albumin adduct burden by LC-HRMS/MS using a biospecimen selected from blood, urine, saliva, and/or a local sample. Comparator data comprises baseline biomarker data for the subject or cohort obtained prior to initiating administration of the embodiment and/or one or more predefined threshold values.

In some embodiments, one or more DNA-associated adduct markers are additionally assessed from DNA obtained from an appropriate biospecimen, including, without limitation, one or more methylation-related DNA adduct markers.

In some embodiments, one or more additional functional readouts are assessed according to a specified assay, formula, baseline, and decision rule. By way of non-limiting example, hemoglobin oxygen affinity (P50) may be measured by a predefined oxygen-dissociation assay and compared with a subject-specific baseline and/or a predefined target range of about 24 mm Hg to about 27 mm Hg.

If, after a defined interval (e.g., about 60 days), total albumin adduct burden will be determined to have decreased by less than about 27% relative to baseline and/or the selected functional readout fails to move toward its predefined target range, subsequent administration may be adjusted by modifying at least one of: (i) probiotic identity, form, or amount; (ii) Component 4 amount or identity; (iii) antioxidant amount; (iv) composition of the nutritional drink; (v) timing of administration relative to meals and/or Component 5; and/or (vi) one or more preparation parameters, including fermentation duration, temperature, pH, light exposure, buffering adjunct amount, and/or order of addition.

If, after the defined interval, total albumin adduct burden will be determined to have decreased by at least about 27% relative to baseline and/or the selected functional readout will be determined to have moved toward the predefined target range, the formulation and/or preparation parameters may be maintained or reduced in a maintenance phase.

### 8.10.4 Prophetic Exemplary Supplement Integration (Beverage)

In one non-limiting embodiment, a PMM will be configured in a beverage format to co-deliver one or more adjunct components with a supplement therapeutic agent (Component 5) in an oral liquid preparation that supports palatability, adherence, and, in certain embodiments, hydration, antioxidant absorption, and/or reduced adverse-event risk. The beverage may be supplied as a ready-to-drink liquid or as a powder for reconstitution with water. In these embodiments, Component 5 is the supplement therapeutic agent, and the overall fermented supplement beverage functions as an oral administration format and/or carrier matrix.

### 8.10.4.a Beverage Composition (Non-Limiting):

(i) a beverage base comprising potable water and, in certain embodiments, one or more electrolyte components selected from sodium, potassium, chloride, citrate, bicarbonate, and combinations thereof, in amounts suitable for oral consumption;
(ii) Component 1: one or more antioxidants and/or polyphenols, including, by way of non-limiting example, resveratrol, ascorbic acid, or combinations thereof;
(iii) Component 2: one or more plant-derived beverage-matrix components, including, by way of non-limiting example, chia seed blended with Irish Sea moss;
(iv) Component 3: one or more probiotic adjuncts provided as viable cells, including, by way of non-limiting example, Saccharomyces boulardii and Bifidobacterium, totaling about 5×10\hat{ }10 CFU;
(v) Component 4: one or more prebiotic and/or fermentation-support components, including, by way of non-limiting example, inulin, sucrose, or combinations thereof, wherein sucrose may be present at about 5 g; and
(vi) Component 5: a supplement therapeutic agent comprising, by way of non-limiting example, a curcumin-containing supplement optionally further comprising black pepper and/or piperine, including, by way of non-limiting example, Turmeric Curcumin with Black Pepper 1000 mg (UPC 850184008091), or an equivalent supplement formulation suitable for ex vivo fermentation prior to administration.

### 8.10.4.b Administration:

In certain embodiments, Component 5 will be combined with all Components 1~4 in the beverage base and fermented ex vivo to form a fermented beverage composition for oral administration.

In other embodiments, Components 1~4 are fermented ex vivo in the beverage base to form a fermented adjunct beverage, and Component 5 with Component 3 and Component 4 will be separately fermented ex vivo prior to administration.

In such embodiments, the separately fermented Component 5 will be admixed with the fermented adjunct beverage immediately before administration, or will be administered concurrently with the fermented adjunct beverage, or will be administered within about 2 hours after completion of fermentation of Component 5.

In some embodiments, the supplement beverage or component 5 will be supplied as a ready-to-drink liquid. In other embodiments, the beverage will be supplied as a powder or sachet for reconstitution with water prior to fermentation and administration.

In certain embodiments, the fermented beverage composition will be administered promptly after completion of fermentation. In other embodiments, the fermented beverage composition will be stored under refrigeration in an appropriate food-safe and/or pharmaceutically suitable container and assigned a beyond-use date based on applicable stability, microbial, and handling considerations.

### 8.10.4.c Monitoring and Iterative Adjustment:

Biomarker data comprising one or more mutagen-related adductomic endpoints will be obtained and processed to determine total adduct burden and/or total albumin adduct burden by LC-MS/MS and/or LC-HRMS/MS using a biospecimen selected from blood, urine, and/or saliva. Comparator data comprises baseline biomarker data for the subject and/or one or more threshold values.

In some embodiments, one or more functional protein metrics are additionally assessed according to a specified assay, formula, baseline, and decision rule, including, by way of non-limiting example, hemoglobin oxygen affinity (P50).

At an initial reassessment interval (e.g., about 14 days), if the adductomic endpoint will be determined to have decreased by less than about 16% relative to baseline and/or one or more functional protein metrics fail to move toward a predefined target value, subsequent administration may be adjusted by modifying at least one of: (i) probiotic identity, form, or amount; (ii) antioxidant and/or polyphenol amount; (iii) beverage administration timing and/or frequency; and/or (iv) one or more preparation parameters, including fermentation duration, temperature, pH, light exposure, buffering adjunct amount, and/or order of addition. If, at the initial reassessment interval, the adductomic endpoint will be determined to have decreased by at least about 29% relative to baseline and/or hemoglobin P50 will be determined to have moved toward a predefined target range, the preparation parameters and/or adjunct amounts may be maintained or reduced in a maintenance phase.

At a later reassessment interval (e.g., about 60 days), if the adductomic endpoint will be determined to have decreased by less than about 17% relative to baseline and/or one or more functional protein metrics fail to move toward a predefined target value, subsequent administration may be further adjusted by modifying at least one of: (i) probiotic identity, form, or amount; (ii) antioxidant and/or polyphenol amount; (iii) beverage administration timing and/or frequency; and/or (iv) one or more preparation parameters, including fermentation duration, temperature, pH, light exposure, buffering adjunct amount, and/or order of addition. If, at the later reassessment interval, the adductomic endpoint will be determined to have decreased by at least about 39% relative to baseline and/or hemoglobin P50 will be determined to have moved toward a target range of about 25 mm Hg to about 29 mm Hg, the preparation parameters and/or adjunct amounts may be maintained or reduced in a maintenance phase.

### 8.11 Quality Systems and Documentation Embodiments (Non-Limiting)

Where regulatory obligations apply, such obligations may be addressed through quality systems, specifications, and controls commensurate with the applicable classification, implementation pathway, and risk profile. Non-limiting examples include identity and potency specifications; stability programs and storage controls; impurity and contaminant limits (including nitrosamine-related controls, where relevant); raw-material qualification; supplier and distributor qualification and oversight; packaging and labeling controls; batch records; complaint handling; root cause analysis; corrective and preventive actions; process controls; quality engineering; and lot/batch traceability. The foregoing will be provided as non-limiting implementation guidance and does not limit the claimed subject matter.

### 8.12 Industrial Applicability

The disclosed compositions, kits, systems, devices, and methods are industrially applicable across multiple sectors, including without limitation:
human pharmaceuticals;
veterinary medicine;
nutraceuticals and dietary supplements;
compounding pharmacies and outsourcing facilities;
clinical, hospital, and outpatient care settings;
contract development and manufacturing organizations (CDMOs); and
distribution, packaging, and medication-adherence solutions.

In some embodiments, the disclosed adjunct compositions and PMM kits, systems, and/or devices may be implemented as add-on and/or co-administered approaches alongside existing therapeutic regimens that will require fermentation of all therapeutic agents. In some embodiments, the adjunct composition is administered in temporal coordination with the fermented primary therapeutic agent to reduce nitrosation risk and/or nitrosamine formation under the use conditions described herein.

In some embodiments, adoption may be facilitated by deploying the PMM and/or PMD as: (i) a separately supplied and readily fermented adjunct product; (ii) a co-packaged product with coordinated fermenting instructions; (iii) a pharmacy-compounded preparation; and/or (iv) a device-associated dosing and/or fermentation system, thereby enabling multiple commercialization pathways for the PMM and PMD depending on patient, cohort, jurisdiction, clinical context, and risk profile. The disclosed embodiments herein may be implemented in a variety of settings, including, without limitation, compounding pharmacies, clinical pharmacy practice, pharmaceutical operations, hospital systems, outpatient clinics, long-term care facilities (LTCFs), skilled nursing facilities, and home-care environments and other similar settings.

### 8.13 Intellectual Property and Claim Scope Considerations

In some embodiments, the present disclosure supports multiple claim sets directed to: (i) adjunct compositions and dosage forms; (ii) methods for reducing and/or mitigating mutagenic burden associated with administration of a therapeutic agent, including embodiments employing fermentation and/or ex vivo preparation steps; (iii) systems configured for dosing, adductomic tracking, monitoring, and iterative protocol adjustment; and (iv) kits comprising separately packaged components and instructions for use. Dependent claims may further recite particular adjunct combinations, dose ranges, administration schedules, threshold and/or comparator criteria for adjustment, fermentation parameters, biomarker endpoints, functional protein metrics, and/or clinical patient subpopulations, thereby providing layered fallback positions across jurisdictions and implementation variants.

### 8.14 Final Statement

While the invention has been described in detail with reference to specific embodiments and examples, those skilled in the art will appreciate that various modifications, additions, substitutions, and combinations may be made without departing from the scope of the invention as defined by the appended claims. All terms used herein shall be interpreted broadly, consistent with their plain meaning and any express definitions provided herein.

What is claimed is:

1. A kit comprising: (a) a therapeutic agent comprising one or more of: (i) an active pharmaceutical ingredient (API); (ii) a marketed or final drug product; or (iii) a supplement administered as the primary therapeutic agent for a defined therapeutic purpose; (b) one or more adjunct components selected from probiotics, prebiotics, antioxidants, buffering agents, chelating agents, coenzymes, plant-derived materials, algae-derived materials, microbial-derived materials, enzymes, vitamins, supplements, and combinations thereof; and (c) instructions directing a user, prior to administration to a subject: (i) to ferment ex vivo, outside the body, the therapeutic agent either (A) in a mixture together with at least one adjunct component or (B) separately from one or more adjunct components; (ii) when the therapeutic agent is fermented according to (c)(i)(B), either (α) to combine the fermented therapeutic agent with one or more adjunct components that have been fermented ex vivo, outside the body, and prior to administration, to form a fermented composition, or (β) to administer the fermented therapeutic agent and the one or more adjunct components that have been fermented ex vivo, outside the body, and prior to administration at separate times relative to one another; (iii) to control, during fermentation of the therapeutic agent and/or the one or more adjunct components, at least one fermentation parameter selected from duration, temperature, pH, light exposure, wavelength, concentration, oxygen level, substrate availability, and order of addition; and (iv) to administer to the subject the mixture of (c)(i)(A), the fermented composition of (c)(ii)(α), or the fermented therapeutic agent and the one or more adjunct components according to (c)(ii)(β).

2. A method of reducing mutagen exposure risk associated with administration of a therapeutic agent, the method comprising: (a) fermenting ex vivo, outside the body, and prior to administration to a subject, the therapeutic agent either (i) in a mixture together with at least one adjunct component or (ii) separately from one or more adjunct components; (b) when the therapeutic agent is fermented according to step (a)(ii), either (α) combining the fermented therapeutic agent with one or more adjunct components that have been fermented ex vivo, outside the body, and prior to administration, to form a fermented composition, or (β) administering the fermented therapeutic agent and the one or more adjunct components that have been fermented ex vivo, outside the body, and prior to administration, at separate times relative to one another; (c) controlling, during fermentation of the therapeutic agent and/or the one or more adjunct components, at least one fermentation parameter selected from duration, temperature, pH, light exposure, wavelength, concentration, oxygen level, substrate availability, and order of addition; and (d) administering to the subject the mixture of step (a)(i), the fermented composition of step (b)(α), or the fermented therapeutic agent and the one or more adjunct components according to step (b)(β).

3. A method of adjusting a fermented therapeutic regimen for a subject, the method comprising: (a) administering to the subject a fermented composition comprising a therapeutic agent and one or more adjunct components, wherein the therapeutic agent was fermented ex vivo, outside the body, and prior to administration; (b) obtaining or receiving biomarker data for the subject, wherein the biomarker data comprises one or more mutagen-related adductomic endpoints; (c) obtaining or receiving one or more functional protein metrics for the subject; (d) comparing the biomarker data and the one or more functional protein metrics to comparator data comprising one or more baseline values and/or one or more threshold values; (e) determining, based on the comparing of step (d), whether a change in the one or more mutagen-related adductomic endpoints is corroborated by a corresponding change or trend in at least one of the one or more functional protein metrics; and (f) determining, based on steps (d) and (e), whether a subsequent administration should be adjusted or maintained and, when adjustment is indicated, modifying at least one of: (i) a selection of the one or more adjunct components, (ii) an amount of at least one adjunct component, (iii) an amount or selection of the therapeutic agent, (iv) timing of administration of at least one of the one or more adjunct components relative to administration of the therapeutic agent, or (v) one or more fermentation parameters.

4. A system for generating regimen instructions for a subject based on biomarker data, the system comprising: (a) an interface and/or communication module configured to receive biomarker data for the subject, wherein the biomarker data comprises one or more mutagen-related adductomic endpoint values and, in some embodiments, one or more functional protein metrics and/or one or more pharmacokinetic (PK) endpoint values and/or pharmacodynamic (PD) endpoint values; (b) a memory storing comparator data; (c) one or more controllers and/or processors configured to compare the biomarker data to the comparator data and, responsive to the comparing, determine an adjustment to a subsequent ex vivo fermentation of a mixture comprising a therapeutic agent and one or more adjunct components, wherein the therapeutic agent is fermented outside the body and prior to administration; and (d) an output configured to provide updated fermentation and/or preparation instructions specifying the adjustment, wherein the updated fermentation and/or preparation instructions specify at least one of: (i) a selection of the one or more adjunct components; (ii) an amount of at least one adjunct component; (iii) an amount of the therapeutic agent; (iv) one or more fermentation parameters selected from fermentation duration, temperature, pH, light exposure, wavelength, concentration, oxygen level, substrate availability, and order of addition; or (v) a timing of administration of the fermented mixture.

5. A system comprising: (a) an organizer comprising one or more removable modules configured to store one or more components of a fermentable mixture; (b) a vessel configured to ferment, outside the body and prior to administration, a composition comprising a therapeutic agent and one or more adjunct components; (c) one or more controllers configured to monitor and/or control at least one fermentation parameter selected from duration, temperature, pH, light exposure, wavelength, concentration, oxygen level, substrate availability, and order of addition; and (d) an interface and/or communication module configured to receive biomarker data for a subject, wherein the biomarker data comprises one or more adductomic endpoints and/or one or more functional protein metrics, and to output updated fermentation and/or preparation instructions based on the received biomarker data and/or clinical data.

6. A composition comprising: (a) a therapeutic agent that has been fermented ex vivo, outside the body, and prior to administration; and (b) one or more adjunct components that have been fermented ex vivo, outside the body, and prior to administration, the one or more adjunct components being selected from probiotics, prebiotics, antioxidants, buffering agents, chelating agents, coenzymes, plant-derived materials, algae-derived materials, microbial-derived materials, enzymes, vitamins, supplements, sugars, and combinations thereof.

7. The kit of claim 1, wherein the one or more adjunct components comprise a probiotic or microbial supplement selected from yeasts of the order Saccharomycesles, bacteria of the class Actinobacteria, bacteria of the class Bacilli, microorganisms of the phylum Cyanobacteria, and combinations thereof.

8. The kit of claim 7, wherein the probiotic or microbial supplement comprises Saccharomyces boulardii.

9. The kit of claim 8, wherein the probiotic or microbial supplement is present at about 10\hat{ }8 to about 10\hat{ }10 colony-forming units or colony-forming-unit equivalents per dose.

10. The kit of claim 1, wherein the one or more adjunct components comprise (i) a plant-derived material derived from a plant belonging to an order selected from the group comprising Ericales, Rosales, Brassicales, Poales, Asparagales, Cucurbitales, Caryophyllales, Asterales, Lamiales, Malpighiales, Fagales, Fabales, Laurales, and Pinales, and/or (ii) an algae-derived material derived from an alga belonging to a phylum selected from the group comprising Ochrophyta, Chlorophyta, Charophyta, and Rhodophyta.

11. The kit of claim 10, wherein the plant-derived material comprises an extract, fraction, or purified constituent enriched in at least one marker constituent selected from total polyphenols, total flavonoids, total anthocyanins, total glucosinolates and/or isothiocyanates, cucurbitacins, phytosterols, squalenes, thiosulfinates, saponins, betalains, terpenes, xanthones, tellimagrandins; pinolenic acid, carotenoids; procyanidins; eugenols, total tocopherols, indoles, tannins, proteolytic enzymes, selenium and/or other minerals, vitamins, and combinations thereof.

12. The kit of claim 1, wherein the one or more adjunct components comprise at least one algae-derived material selected from brown algae-derived material, red algae-derived material, green algae-derived material, and combinations thereof.

13. The kit of claim 12, wherein the algae-derived material comprises an extract, fraction, or purified constituent enriched in at least one marker constituent selected from total sulfated polysaccharides, phycobiliproteins, porphyran-type sulfated polysaccharides, phycoerythrin-related proteins, carotenoids, tocopherols, polyphenols and combinations thereof.

14. The kit of claim 1, wherein the one or more adjunct components comprise a buffering agent configured to adjust the pH of the mixture during fermentation to a range selected from about pH 2.5 to about pH 10.5, about pH 2.5 to about pH 8, about pH 3.0 to about pH 7.5, or about pH 3.5 to about pH 6.5.

15. The kit of claim 1, wherein the one or more adjunct components comprise a chelating agent configured to sequester one or more metal ions associated with catalysis of nitrosation and/or mutagen formation.

16. The kit of claim 1, wherein the therapeutic agent and the one or more adjunct components are provided in a dosage form selected from a liquid, suspension, powder, capsule, tablet, sachet, dispersible granule, cream, gel, ointment, foam, paste, oil, lotion, or spray.

17. The kit of claim 1, wherein the instructions specify one or more fermentation parameters selected from fermentation duration, temperature, pH, light exposure, wavelength, concentration, oxygen level, substrate availability, and order of addition.

18. The kit of claim 1, wherein a mass ratio of the therapeutic agent to a total mass of the one or more adjunct components is from about 1 g:0.000001 g to about 1 g:1000 g.

19. The kit of claim 18, wherein the mass ratio is from about 1 g:0.00001 g to about 1 g:1000 g.

20. The kit of claim 19, wherein the mass ratio is from about 1 g:0.001 g to about 1 g:100 g.

21. The method of claim 2, wherein fermentation duration is from about 1 minute to about 24 hours.

22. The method of claim 2, wherein fermentation temperature is from about 15° C. to about 40° C.

23. The method of claim 2, wherein pH of the mixture during fermentation is within a range selected from about pH 2.5 to about pH 10.5, about pH 2.5 to about pH 8, about pH 3.0 to about pH 7.5, or about pH 3.5 to about pH 6.5.

24. The method of claim 2, wherein fermentation further comprises controlled light exposure at one or more wavelengths selected from ultraviolet, white, blue, and red light.

25. The method of claim 2, wherein the subject is a human.

26. The method of claim 2, wherein the subject is a non-human animal.

27. The method of claim 3, wherein the comparator data comprises at least one of: (i) subject-specific baseline biomarker data; (ii) a threshold value derived from a matched-cohort comparator; (iii) a threshold value derived from a population reference range; or (iv) a threshold value.

28. The method of claim 3, wherein the comparing comprises determining, by a targeted and/or untargeted adductomics assay, a total adduct burden for the subject relative to the comparator data.

29. The method of claim 28, wherein the total adduct burden comprises an albumin adduct burden.

30. The method of claim 29, wherein, in a human subject, the albumin adduct burden comprises a human serum albumin (HSA) adduct burden.

31. The method of claim 30, wherein the HSA adduct burden decreases to at most about 0.1 μmol adduct per mg HSA.

32. The method of claim 3, wherein the comparator data comprises a subject-specific baseline value, and wherein the subsequent administration is adjusted when at least one adductomic endpoint decreases by less than a predefined percentage relative to the subject-specific baseline value.

33. The method of claim 32, wherein the subsequent administration is maintained when (i) the at least one adductomic endpoint decreases by at least the predefined percentage relative to the subject-specific baseline value and/or reaches a threshold value, and (ii) at least one functional protein metric moves toward a predefined target value relative to the subject-specific baseline value.

34. The method of claim 3, wherein the one or more functional protein metrics are selected from hemoglobin oxygen affinity (P50), INR, whole-blood clotting time, a relationship between change in ferritin and/or transferrin with change in hemoglobin over a defined interval, discordance between HbA1c-derived estimated average glucose and longitudinal averages of directly measured blood glucose values over a defined interval, a predefined vitamin B12/intrinsic factor antibody relationship metric, and combinations thereof.

35. The method of claim 34, wherein the subsequent administration is maintained when albumin adduct burden decreases by at least about 19% relative to baseline and hemoglobin P50 trends toward about 25 mm Hg±2 mm Hg.

36. The method of claim 3, further comprising obtaining or receiving pharmacokinetic data, pharmacodynamic data, and/or clinical data for the subject, and adjusting the subsequent administration based further on the pharmacokinetic data, the pharmacodynamic data, and/or the clinical data, wherein the clinical data comprises at least one of medication regimen data, diagnosis data, laboratory data, allergy data, dietary data, lifestyle data, tolerability and adverse effects data, or therapeutic-response data.

37. The system of claim 4, wherein the updated fermentation and/or preparation instructions are provided in at least one format selected from printed matter, an electronic message, an audio communication, and data stored on a non-transitory computer-readable medium.

38. The system of claim 4, wherein the interface and/or communication module is further configured to receive clinical data comprising at least one of medication regimen data, diagnosis data, laboratory data, allergy data, dietary data, or lifestyle data, to generate the updated fermentation and/or preparation instructions based on the biomarker data and/or the clinical data, and to transmit the updated fermentation and/or preparation instructions to a medication therapy management module and/or a comprehensive medication management module.

39. The system of claim 5, wherein the one or more removable modules are detachably connected to the organizer to permit separate storage and subsequent reattachment.

40. The system of claim 5, wherein the vessel comprises a lid, a light source configured to emit one or more wavelengths selected from ultraviolet, white, blue, and red light, and a temperature monitor.

41. The method of claim 2, wherein the one or more adjunct components comprise: (a) about 50 mg ascorbic acid; (b) a plant- and/or algae-derived component comprising about 20 g chia seed material (Salvia hispanica) and at least one of about 40 g kiwi material (Actinidia deliciosa) or about 15 g Irish sea moss; (c) about 250 mg Saccharomyces boulardii; and (d) about 1 g sucrose.

42. The method of claim 41, wherein the therapeutic agent comprises a marketed drug product comprising an API susceptible to formation of a nitrosamine drug substance-related impurity, and wherein fermenting the mixture ex vivo comprises: (a) blending the plant- and/or algae-derived component with water to form a slurry; (b) adding potable water at about 28° C. to about 33° C. to a fermenter to a predetermined fill level; (c) introducing the ascorbic acid, the Saccharomyces boulardii, the sucrose, and the therapeutic agent into the fermenter; (d) fermenting for about 3 minutes at about 28° C. to about 33° C.; (e) adding the slurry formed in step (a) and mixing; and (f) administering the mixed contents to the subject according to a prescribed regimen.

43. The method of claim 42, further comprising scheduling and/or confirming subsequent biomarker collection for adductomic assessment and/or assessment of one or more functional protein metrics.

44. The method of claim 2, wherein the therapeutic agent comprises diclofenac, and wherein combining comprises forming a fermentable mixture comprising: (a) a topical diclofenac drug product providing diclofenac at about 0.1% to about 5% (w/w); (b) tocopherol and/or an ascorbic acid derivative at about 0.01% to about 5% (w/w); (c) optionally an algae-derived adjunct comprising Irish sea moss; (d) a probiotic adjunct comprising Saccharomyces boulardii and/or Bifidobacterium at about 10\hat{ }6 to about 10\hat{ }11 CFU-equivalents per unit dose or per gram of mixture; and (e) optionally sucrose, inulin, or another fermentable carbohydrate at about 0.1% to about 10% (w/w).

45. The method of claim 44, wherein fermenting the mixture ex vivo comprises fermenting the mixture at about 20° C. to about 37° C. for about 2 minutes to about 24 hours and at a pH of about 4.0 to about 7.5, and wherein administering comprises topically applying the fermented diclofenac composition to the subject.

46. The method of claim 45, further comprising scheduling and/or confirming subsequent biomarker collection for adductomic assessment and/or assessment of one or more functional protein metrics.

47. The kit of claim 1, wherein the instructions direct the user to ferment the therapeutic agent separately from at least one adjunct component and to administer the fermented therapeutic agent and the at least one adjunct component that has been fermented ex vivo, outside the body, and prior to administration at separate times relative to one another, with the at least one adjunct component being administered before or after administration of the fermented therapeutic agent.

48. The kit of claim 1, wherein the therapeutic agent and at least one adjunct component are provided in separate sets or packages, and wherein the instructions specify patient-specific or cohort-specific fermentation and administration directions for the separate sets or packages.

49. The kit of claim 1, wherein the instructions direct the user to ferment the therapeutic agent separately from at least one adjunct component and then combine the fermented therapeutic agent with one or more adjunct components that have been fermented ex vivo, outside the body, and prior to administration, to form a fermented composition.

50. The composition of claim 6, wherein the therapeutic agent is fermented ex vivo together with at least one of the adjunct components prior to administration.

51. The method of claim 36, wherein the clinical data further comprise one or more tolerability endpoints selected from hospitalization, adverse-event incidence, adverse-event severity grade, dose interruption, dose reduction, discontinuation, local irritation, gastrointestinal intolerance, sedation, dizziness, orthostatic symptoms, palatability-related nonadherence, and combinations thereof.

52. The method of claim 51, wherein the subsequent administration is adjusted to reduce the one or more tolerability endpoints while maintaining the therapeutic benefit of the therapeutic agent.

53. The system of claim 4, wherein the biomarker data and/or clinical data further comprise one or more tolerability endpoints, and the updated fermentation and/or preparation instructions are further configured to improve a tolerability endpoint while maintaining the therapeutic benefit of the therapeutic agent.
